(19) United States
(12) Patent Application Publication
(10) Pub. No.: US 2011/0319352 A1
(43) Pub. Date: Dec. 29, 2011

(54) Compounds That Bind Oxysterol Binding Proteins, and Methods of Use Thereof

(75) Inventors: Matthew D. Shair, Lexington, MA (US); Anthony Burgett, Arlington, MA (US); D. Ryan Anderson, Cambridge, MA (US)

(73) Assignee: President and Fellows of Harvard College, Cambridge, MA (US)

(21) Appl. No.: 13/139,196

(22) PCT Filed: Dec. 11, 2009
(86) PCT No.: PCT/US2009/067689

§ 371 Date: Sep. 12, 2011

Related U.S. Application Data
(60) Provisional application No. 61/121,671, filed on Dec. 11, 2008.

(51) Int. Cl. A61K 31/704; A61K 31/352; A61P 35/00; A61P 9/10; A61P 25/28; A61K 31/58; A61K 31/202

(52) U.S. Cl. 514/26; 514/173; 514/454; 514/559

(57) ABSTRACT

The invention relates in part to the discovery that certain CRAMs, such as schweinfurthin A, target OSBPs (a family of oxysterol binding proteins). Because OSBPs have been shown to be integral to atherosclerosis and Alzheimer's disease (AD), one aspect of the invention relates to the use of CRAMs, or a pharmaceutically acceptable salt, biologically active metabolite, solvate, hydrate, prodrug, enantiomer or stereoisomer thereof, in the treatment and/or prevention of atherosclerosis, Alzheimer's disease and related disorders. Another aspect of the invention relates to novel derivatives of CRAMs, or a pharmaceutically acceptable salt, biologically active metabolite, solvate, hydrate, prodrug, enantiomer or stereoisomer thereof, for the treatment and/or prevention of atherosclerosis, Alzheimer's disease and related disorders. Another aspect of the invention relates to the use of an immobilized CRAMs, such as OSW-I, to aid in screening of compounds to identify additional OSBP binders. Other aspects of the invention relate to the use of CRAMs to treat cancer, such as p21-deficient cancers.

### RELATED APPLICATION

This application claims the benefit of priority to U.S. Provisional Patent Application Ser. No. 61/121,671, filed Dec. 11, 2008; the contents of which are hereby incorporated by reference.

### BACKGROUND

### CRAMs (Cephalostatin and Related Antiproliferative Molecules)

Cephalostatin 1 (1) [1], OSW-1 (2) [2,3], ritterazine B (3) [4], schweinfurthin A (4) [5], and (−)-stellettin E (5) [6] are natural products that are potently cytotoxic to selected human cancer cell lines (FIG. 1). These molecules also have highly similar cytotoxicity patterns against the 60 cultured human cancer cell lines evaluated by the National Cancer Institute (NCI-60) [7], strongly suggesting that they share a cellular target or act by similar mechanisms. Compounds with Pearson correlation coefficients (p values) greater than 0.6 (1.0 is a perfect match) by COMPARE analysis are considered mechanistically related [7, 8] (see FIG. 1 for p values between 2-4 and 1). Since 1-5 are likely related by their mechanism, they are referred to herein as CRAMs (Cephalostatin and Related Antiproliferative Molecules). Also, since the cellular targets and mechanisms of all CRAMs may be related, studying one compound may lead to an understanding of all CRAMs.

Despite significant interest in the antiproliferative activity of CRAMs, their cellular targets and mechanisms have not been elucidated. Moreover, COMPARE analysis between more than 40,000 compounds in the NCI collection and CRAMs revealed little or no similarity, suggesting that the mechanisms and cellular targets of CRAMs may be unique. [9]

### Cytoplasmic Oxysterol Receptors

In the early 1980s the hypothesis was advanced that cholesterol synthesis is regulated via an oxysterol binding protein; i.e., a cytosolic component that bound to oxysterols. Today two protein families have been described which are known to bind oxysterols: The liver X receptors (LXRs); and the protein family described as cytoplasmic oxysterol receptors, termed oxysterol binding proteins (OSBPs) or OSBP related proteins, ORPs.

OSBP (oxysterol-binding protein) is a protein found in eukaryotes that was first identified based on its high affinity for oxysterols, especially 25-hydroxycholesterol (K_{d}=37 nM). It is the founding member of a family of evolutionarily conserved proteins, 12 are found in mammals (see FIG. 6). OSBP is a 89 kD protein comprising a sterol-binding domain and several other domains involved in protein-protein and protein-lipid interactions. These domains include a pleckstrin homology (PH) domain which localizes proteins to phosphatidylinositol-containing membranes and an FFAT domain, which is an ER-localizing domain. [20, 21] OSBP does not exhibit enzymatic activity. [20, 21] Although an understanding of its cellular function remains incomplete, studies in the last few years reveal that OSBP is a sterol sensor that can exert control over key signaling pathways. [20, 21]

OSBP is involved in 25-hydroxycholesterol (25-OHC)-induced increase of sphingomyelin synthesis through activation of ceramide transfer protein (CERT) (FIG. 13, i). [22] Sphingomyelin is a phospholipid component of cell membranes that is synthesized from ceramide and phosphatidylcholine. Some inducers of apoptosis such as Fas ligands do so by activating sphingomyelinase causing sphingomyelin hydrolysis and generation of ceramide, which is known to cause apoptosis. [23] Binding of 25-OHC to OSBP causes, indirectly, an increase in transport of ceramide from the ER to the Golgi, where sphingomyelin synthase 1 is located. [24] This leads to an increase of sphingomyelin synthesis. [24] It is thought that OSBP and CERT work in concert to coordinate cholesterol and sphingomyelin levels in cells, which is important to maintain proper membrane structure. Importantly, an OSBP knockout mouse was reported to be embryonic lethal (mentioned in [25]). This suggests although OSBP is required for animal development, cell lines can survive with OSBP expression ablated.

A second activity of OSBP was revealed when it was discovered that caveolae of the plasma membrane that were deprived of cholesterol or treated with 25-OHC caused differential phosphorylation states of ERK1/2 (extracellular signal-regulated kinase 1 and 2). [26] OSBP was found to be the sterol receptor mediating this event. [27] Upon binding cholesterol, OSBP forms a ternary complex with two phosphatases, PP2A (a serine/threonine phosphatase) and HePTP (a tyrosine phosphatase), which then inactivates ERK1/2 through dephosphorylation (FIG. 13, iia). [27] Conversely, addition of 25-hydroxycholesterol (25-OHC) or depletion of cholesterol disassembles this complex, leading to ERK1/2 hyperphosphorylation (FIG. 13, iib). [27] ERK1/2 is a component of the MAPK/ERK signaling pathway, which is responsible for linking growth factor signaling with cellular responses including cell division. [28] The discovery that OSBP can control ERK1/2 activation is exciting since it shows that OSBP can link sterol binding to key signaling pathways in the cell.

It has also been reported that OSBP can control up-regulation of profilin-1 through activation of the JAK-STAT pathway in response to 25-OHC and 7-ketocholesterol. [29] However, these effects have only been observed in specific endothelial cells that may not represent roles for OSBP in cancer cells.

As the oxysterol-binding protein/oxysterol-binding protein-related protein family has been implicated in lipid transport and metabolism, vesicle trafficking and cell signaling, this protein family contains attractive targets for the treatment of a range of disorders.

In addition to OSBP, mammals express 11 other OSBP-related proteins referred to as ORPs (FIG. 4A). [20, 21] Like OSBP, each ORP contains a sterol-binding domain, although beyond that there is wide variation in ORP configuration. [20, 21] The sterol-binding domain of OSBP/ORPs is conserved among the family of 12 proteins, but it is not similar to sterol-binding domains of other proteins such as LXR [30], Insig, [31] or NPC-1 [32]. The ORPs are less well characterized than OSBP, although they have been implicated in vesicular traffic, cell signaling, and possibly sterol transport. [20, 21] Currently, their cellular functions are not well understood, although it is believed that they are playing important cellular roles since they are expressed ubiquitously and they are evolutionarily conserved. [20, 21] They have been implicated in atherosclerosis [33-35] and possibly cancer [36, 37]. For instance, overexpression of ORP1L leads to atherosclerotic lesions in mice. [33] Additionally, atherosclerotic lesions were found to contain up-regulated ORP8 [34], and ORP9 has been identified as a new therapeutic target in raising HDL levels. [38] Knockdown of OSBP or ORP8 led to enhanced levels of cholesterol efflux upon LXR agonism. [34, 35] Increased cholesterol efflux is a therapeutic approach in the treatment of atherosclerosis [39, 40], suggesting that ORPs may be atherosclerosis drug targets. In yeast, there are 7 Osh proteins (ORP homologs). Interestingly, yeast can survive with any 6 of their 7 Osh proteins deleted, but not all 7. [40]

The only structural information on this class of proteins is an X-ray crystal structure of Osh4, a yeast homolog of ORP4S. [25] The structure of Osh4 was solved bound to multiple sterols. [25] Sterol binding induces closure of an α-helical lid [25] leading to dramatic conformational changes in the protein. Osh4 is homologous to the OSBP/ORP sterol binding domain. [25] Extrapolating from the effects of sterol binding on Osh4, OSBP/ORPs ligand binding could induce conformational changes and alter the proteins' function. [25]

### Atherosclerosis

Atherosclerosis results from high serum cholesterol levels (hypercholesterolemia) which leads to the accumulation of cholesterol in arterial walls. The plaques that characterize atherosclerosis inhibit blood flow and promote clot formation, and can ultimately cause death or severe disability via heart attacks and/or stroke.

OSBPs may be useful in the treatment of atherosclerosis. For example, it is known that mammalian oxysterol-binding protein-1 (OSBP1) binds oxygenated derivatives of cholesterol and mediates sterol and phospholipid synthesis. In addition, it has been recently shown that mammalian oxysterol-binding protein-related protein 8 (ORP8) acts as a negative regulator of ABCA1 expression and macrophage cholesterol efflux, and thus may modulate the development of atherosclerosis. Yan, D. et al. “OSBP-related Protein 8 (ORP8) Suppresses ABCA1 Expression and Cholesterol Efflux from Macrophages,” J. Biol. Chem. 2008, 283, 332-340.

### Alzheimer's Disease

Alzheimer's disease is a common, chronic neurodegenerative disease that often leads to dementia and death, which is characterized by a progressive loss of memory and sometimes severe behavioral abnormalities, as well as an impairment of other cognitive functions. It ranks as the fourth leading cause of death in industrialized societies after heart disease, cancer, and stroke. The incidence of Alzheimer's disease is high, with an estimated 2.5 to 4 million patients affected in the United States and perhaps 17 to 25 million worldwide. Moreover, the number of sufferers is expected to grow as the population ages. A characteristic feature of Alzheimer's disease is the presence of large numbers of insoluble deposits, known as amyloid plaques, in the brains of those affected. Autopsies have shown that amyloid plaques are found in the brains of virtually all Alzheimer's patients and that the degree of amyloid plaque deposition often correlates with the degree of dementia. While some opinion holds that amyloid plaques are a late stage by-product of the disease process, the majority view is that amyloid plaques and/or soluble aggregates of amyloid peptides are more likely to be intimately, and perhaps causally, involved in Alzheimer's disease. Because there is no cure for Alzheimer's disease, managing the disease usually involves medications to control symptoms, in combination with various non-drug strategies designed to ease the suffering of the person afflicted and that of his or her family and caregivers. Unfortunately, not all patients with Alzheimer's disease are responsive to the currently available therapies.

OSBP1 has been shown to modulate processing and trafficking of the amyloid precursor protein. Laitinen, S. et al. “ORP2, a Homolog of Oxysterol Binding Protein, Regulates Cellular Cholesterol Metabolism,” Journal of Lipid Research 2002, 43, 245-255. These results suggest that OSBP1 could play a role in linking cholesterol metabolism with intracellular amyloid production, and, more importantly, indicate that OSBP1 could provide an alternative target for a directed therapeutic for human maladies including Alzheimer's disease and atherosclerosis.

### SUMMARY

The invention relates in part to the discovery that certain CRAMs, such as schweinfurthin A, target OSBPs (a family of oxysterol binding proteins). Because OSBPs have been shown to be integral to atherosclerosis and Alzheimer's disease (AD), one aspect of the invention relates to the use of CRAMs, or a pharmaceutically acceptable salt, biologically active metabolite, solvate, hydrate, prodrug, enantiomer or stereoisomer thereof, in the treatment and/or prevention of atherosclerosis, Alzheimer's disease, or related disorders. Another aspect of the invention relates to novel derivatives of CRAMs, or a pharmaceutically acceptable salt, biologically active metabolite, solvate, hydrate, prodrug, enantiomer or stereoisomer thereof, for the treatment and/or prevention of atherosclerosis, Alzheimer's disease, or related disorders. Another aspect of the invention relates to the use of an immobilized CRAMs, such as OSW-1, to aid in screening of compounds to identify additional OSBP binders. Other aspects of the invention relate to the use of CRAMs to treat cancer, such as p21-deficient cancers.

### BRIEF DESCRIPTION OF THE FIGURES

FIG. 1 depicts cephalostatin 1 (1), OSW-1 (2), ritterazine B (3), schweinfurthin A (4) and B, and (−)-stellettin E (5), and their respective GI₅₀ (NCI-60) values.

FIG. 2 depicts schweinfurthins A, B, C and D.

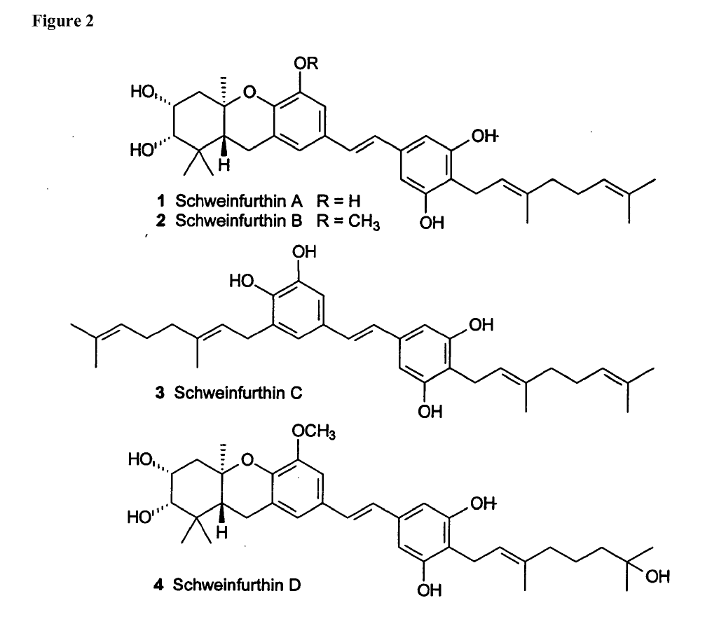

FIG. 3 depicts the results of a OSW-1 pulldown experiment from HeLa cytosol. Protocol: (1) prepare S-100 HeLa cytosol lysate; (2) preincubate competition lysate with 15 mM OSW-1 for 1 hr; (3) incubate 50 mL of AB-9 and AB-11 Resin (˜16 mmol AB-9/mL resin) with 50 mg protein overnight; and (4) wash resin and elute in sample buffer.

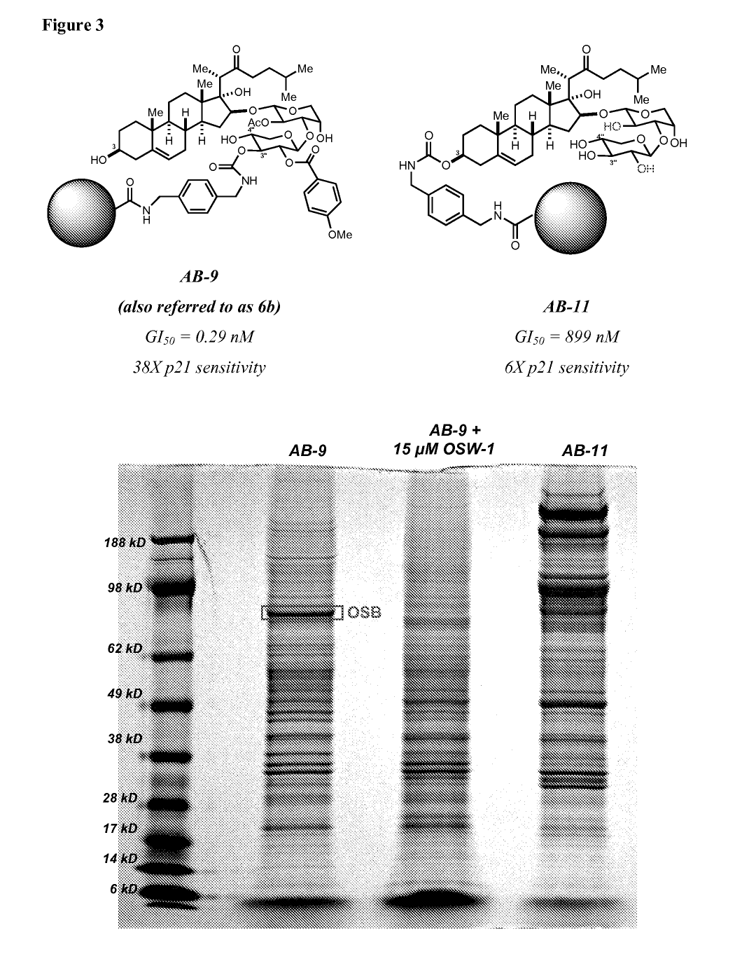

FIG. 4 depicts the structure of oxysterol binding protein (OSBP) and cholesterol bound thereto; see Im, Y. J. et al, “Structural mechanism for sterol sensing and transport by OSBP-related proteins,” Nature 2005, 437, 154-158.

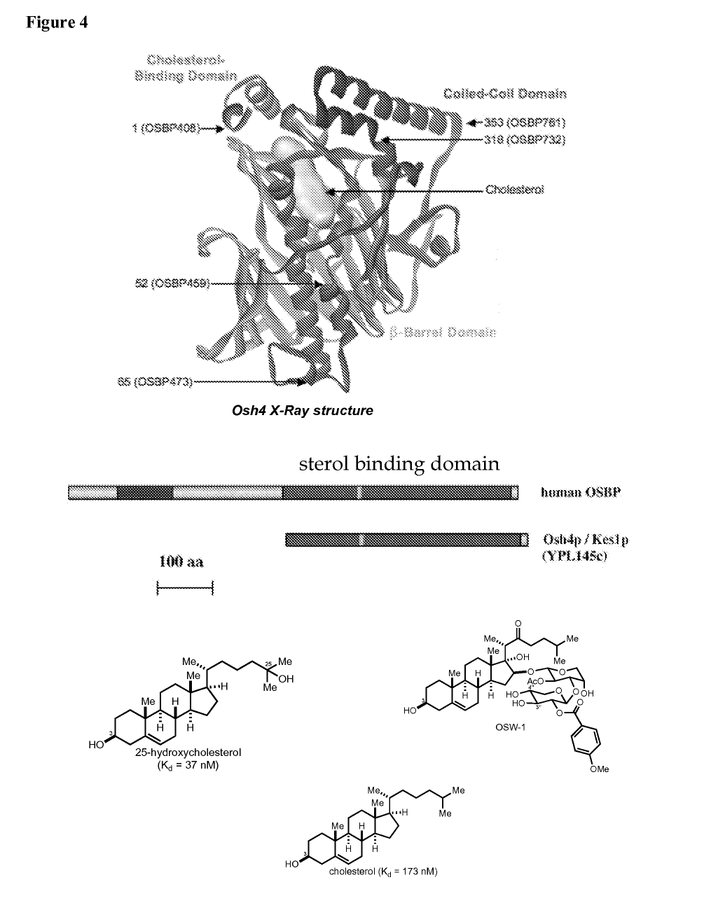

FIG. 5 depicts a gel showing sites where OSBP-related proteins are found in vivo; see Jaworski, C. J. et al. “A Family of 12 Human Genes Containing Oxysterol-Binding Domains,” Genomics 2001, 78(3), 185-196.

FIG. 6 depicts a comparison of OSBP-related proteins, illustrating that ORPs have diverse localizations and functions and multiple splice variants; see Lehto, M. and Olkkonen, V. M. “The OSBP-related proteins: a novel protein family involved in vesicle transport, cellular lipid metabolism, and cell signaling,” BBA 2003, 1631, 1-11.

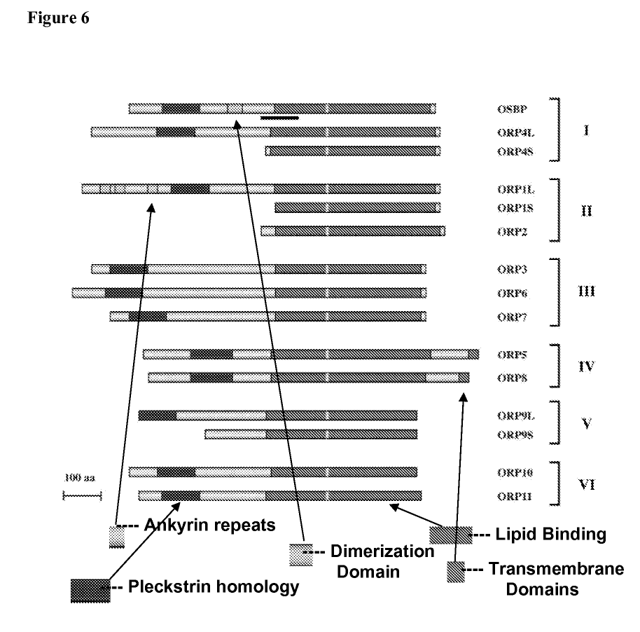

FIG. 7 depicts a diagram showing the possible cellular roles of OSBP. See also FIG. 13.

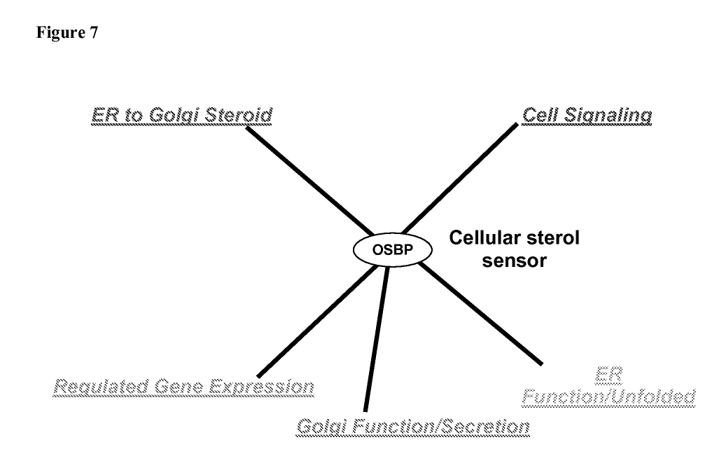

FIG. 8 depicts results of a knockdown of OSBP which sensitizes cells to OSW-1. Specifically, HCT-116 p21−/− cells OSBP knocked down by 89% with shRNA. OSBP knockdown HCT-116 p21−/− cells are 5.7× more sensitive to OSW-1. “Bref A” is brefeldin A.

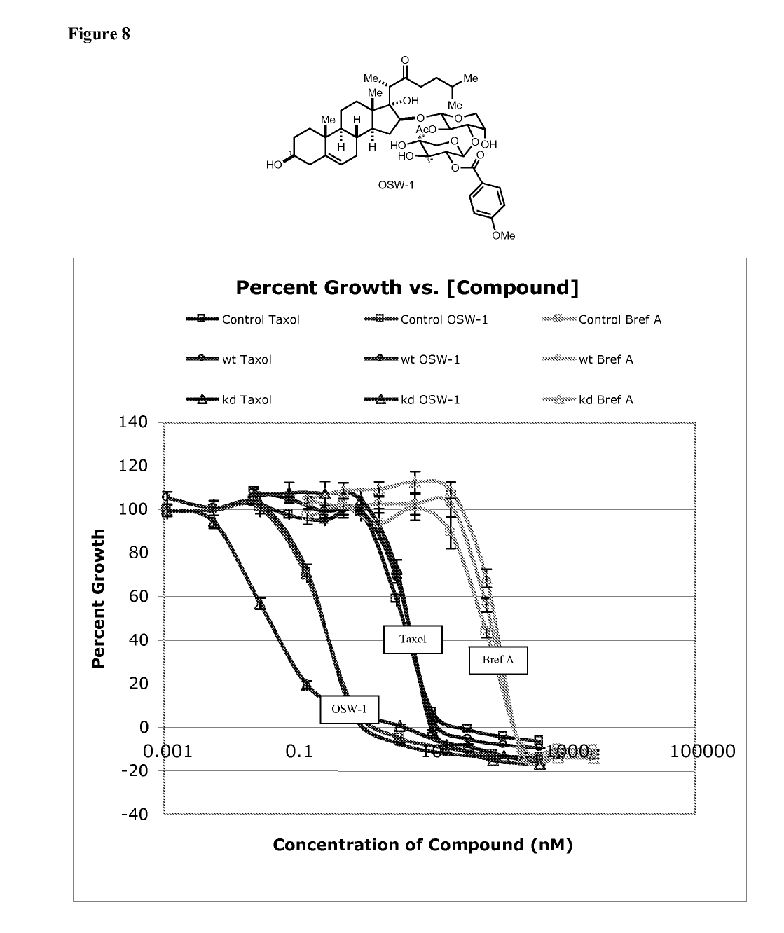

FIG. 9 depicts results of a knockdown of OSBP which sensitizes cells to OSW-1. Specifically, HeLa cells OSBP knocked down by 82% with shRNA. OSBP knockdown HeLa cells are 4.8× more sensitive to OSW-1. “Bref A” is brefeldin A.

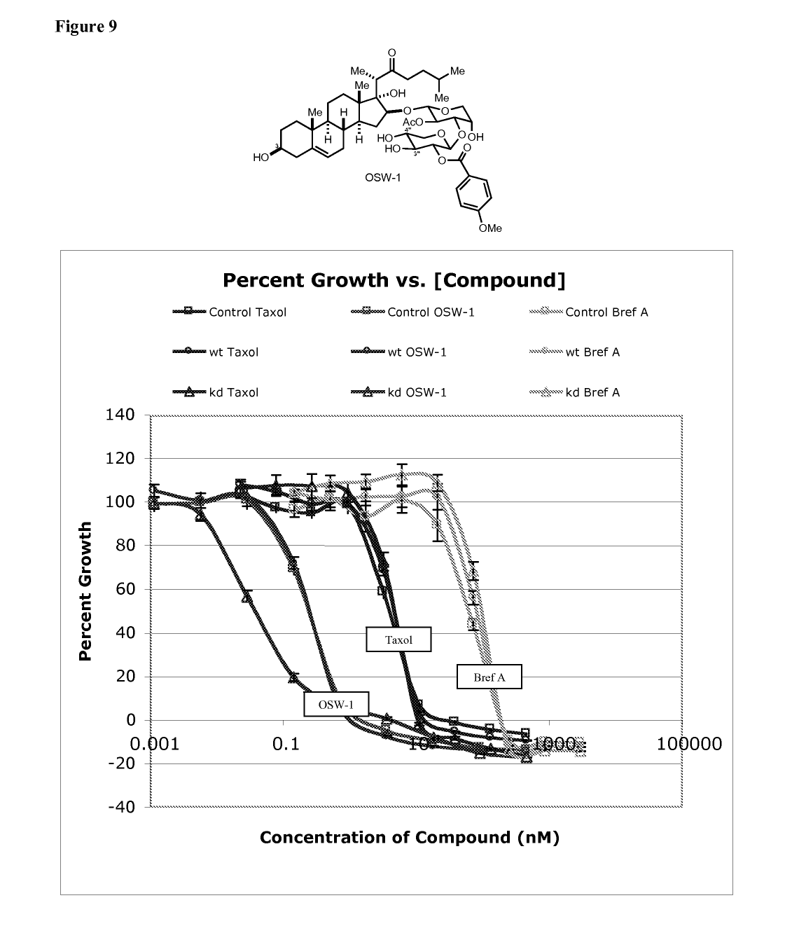

FIG. 10 depicts results of knockdown of OSBP which sensitizes cells to schweinfurthin A and ritterazine B. OSBP knockdown sensitizes cells to OSW-1, schweinfurthin A, and ritterazine B, but not other cytotoxics.

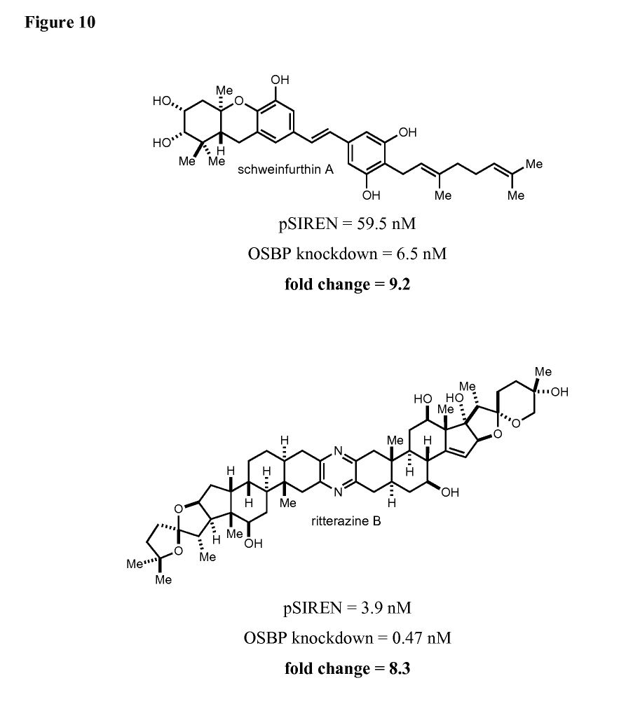

FIG. 11 depicts results showing that OSW-1, but not other cytotoxics, is selectively growth inhibitory to p21-deficient cells. Interestingly, p21 differential cytotoxicity does not appear to be exploited by any current anti-cancer therapies.

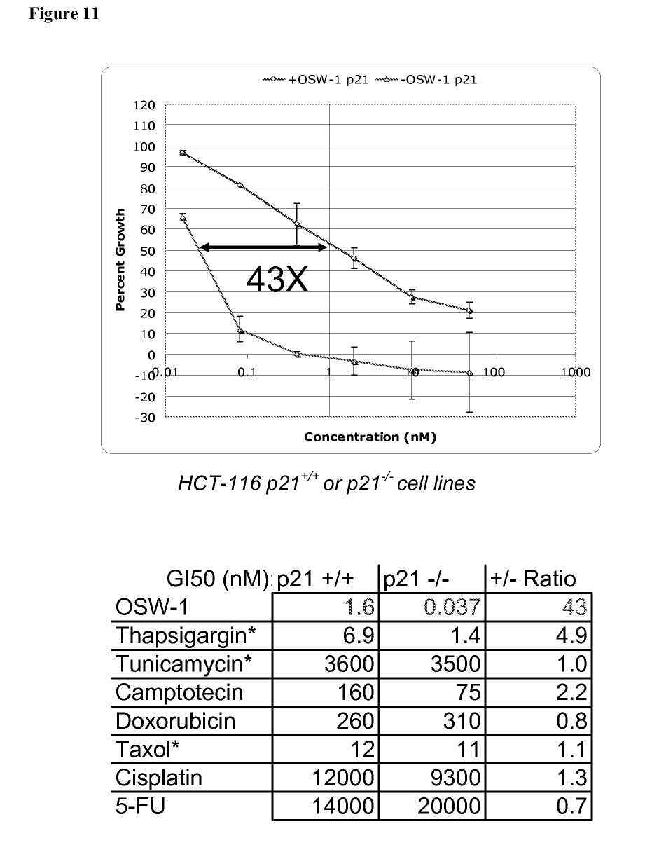

FIG. 12 depicts a graph showing that ritterazine B and schweinfurthin A are also selectively growth inhibitory for HCT-116 cells lacking p21.

FIG. 13 depicts selected activities of OSBP.

FIG. 14 depicts GI₅₀ values of CRAMs and other toxins in HCT-116 p21^{+/+} and p21^{-/-} cells (48 h). Values are mean±SD of 3 individual experiments each performed in sextublicate or from a single experiment performed in sextublicate. Values for (−)stellettin E were reported in the literature (see Beutler J A, Shoemaker R H, Johnson T, Boyd M R. “Cytotoxic geranyl stilbenes from Macaranga schweinfurthii,” J. Nat. Prod. 1998, 61(12), 1509-12).

FIG. 15 depicts structures of OSW-1 analogs carrying carbamate linker, affinity matrix, and affinity chromatography experiment; band 1 is OSBP.

FIG. 16 depicts structures of OSW-1 analogs and GI₅₀ values for CRAMs and OSW-1 analogs in three cancer cell lines at 72 h.

FIG. 17 depicts CRAM cytotoxicity in shRNA OSBP knockdown cell lines. A: Dose curves from a representative experiment. B: GI₅₀ values of CRAMs and control compounds in HCT-116 p21−/− shNT and shOSBP cells and densitometric analysis of OSBP-levels. shNT=non-targeting shRNA, shOSBP=OSBP-targeting shRNA; * P=0.0006 (n=4), ** P=0.0002 (n=3), *** P=0.0003 (n=3), and **** P≦0.0001 (n=7) compared to shNT-treated cells.

FIG. 18 depicts inhibition of binding of 20 nM [³H]-25-OHC to OSBP- and to ORP4-myc-his by CRAMs.

FIG. 19 depicts the correlation between inhibition-constants (Kᵢ) and toxicity (GI₅₀) of CRAMs and OSW-1 analogs in the HCT-116 p21^{-/-} cell line.

FIG. 20 depicts [³H]-serine pulselabel experiment in CHO-K1 cells monitoring label incorporation into sphingomyelin; * P≦0.01 (n=3) and ** P=0.03 (n=3) compared to vehicle-treated cells.

FIG. 21 depicts Western blot and densitometry analysis of pERK1/2 in HCT-116 cells during treatment with 50 nM cephalostatin 1. 25-OHC (20 μM) and okadaic acid (OKA, 10 nM) treatments were for 12 h and 24 h, respectively.

FIG. 22 depicts OSBP localization in HCT-116 p21^{+/+} cells imaged by immunofluorescence confocal microscopy. Immunofluorescence was used to image OSBP and the trans-Golgi protein p230 after compound exposure for 4 h. Colocalization of OSBP and p230 was labeled with Hoescht 33342.

FIG. 23 depicts graphs showing the results of cells treated with 1 or 2 induced a time-dependent reduction in OSBP protein levels.

FIG. 24 depicts proposed synthesises of: (A) Isotope-labeled OSW-1, (B) Isotope-labeled Cephalostatin 1: (a) 10, 11, TMSOTf, CH₂Cl₂; (b) AcOH/H₂O; (c) DDQ, H₂O; (d) Pd/C, D₂ or T₂, ⁱPr₂NEt, THF; (e) NaH, HMPA, H₂O; (f) DMSO, oxalyl chloride, CH₂Cl₂; (g) ²H₄BNa/³H₄BNa, MeOH; (h) TBAF, THF.

FIG. 25 depicts a proposed synthesis of cephalostatin 1 analogs: (a) TBAF, THF, rt; (b) TBAF, THF, reflux; (c) isopropenyl acetate, 1,3-dichlorotetrabutyldistannoxane; (d) TBSCl, imidazole, DMF; (e) K₂CO₃, MeOH.

FIG. 26 depicts the biochemical interactions underlying a scintillation proximity binding assay.

### DETAILED DESCRIPTION

Remarkably, we have discovered that CRAMs (Cephalostatin and Related Antiproliferative Molecules) are high affinity ligands of OSBP and ORP4, and that CRAMs perturb several OSBP activities in cells. In addition, the results presented herein suggest that OSBP/ORPs are the targets mediating the cytotoxicity of CRAMs. The finding that OSBP/ORPs are required for cancer cell survival suggests that these proteins may be promising targets for cancer chemotherapy.

Also provided herein are proposed methods to clarify which OSBP/ORPs are the targets mediating cytotoxicity of CRAMs and, secondly, to examine why CRAMs are cytotoxic to cancer cells.

In addition, since OSBPs have been shown to be critical to atherosclerosis and Alzheimer's disease (AD), another aspect of the invention relates to the use of CRAMs, and analogs thereof, for the treatment and/or prevention of atherosclerosis, Alzheimer's disease, or related disorders.

### Cephalostatin 1 and Ritterazine B

Cephalostatins are a group of complex steroidal pyrazine alkaloids, which were isolated from the sea worm Cephalodiscus gilchristi. They represent cytotoxins that are highly effective against the PS cell line (ED₅₀ of 10⁻⁷-10⁻⁹ μg/mL) and are anti-tumor agents. They are natural marine products that occur rarely, however, and are available only in extremely small amounts. For example, only 139 mg of cephalostatin 1 and a total of 272 mg of other cephalostatins could be isolated from 166 kg of Cephalodiscus gilchristi (tubular worms 5 mm long). All of the cephalostains (such as cephalostatin 1, cephalostatin 2, cephalostatin 3, cephalostatin 4, cephalostatin 5, cephalostatin 6, cephalostatin 7, 25′-epi-cephalostatin 7, 20-epi-cephalostatin 7, cephalostatin 8, cephalostatin 9, cephalostatin 10, cephalostatin 11, cephalostatin 12, cephalostatin 13, cephalostatin 14, cephalostatin 15, cephalostatin 16, cephalostatin 17, cephalostatin 18, and cephalostatin 19) are intended to be encompased.

In addition to its unusual COMPARE profile, another indication that the cellular target and mechanism of cephalostatin 1 (1), and likely all CRAMs, is unique was the report that cephalostatin 1 induces apoptosis without formation of the apoptosome and without release of cytochrome c from the mitochondria, both events that occur with most cytotoxic anti-cancer small molecules. [10, 11] Treatment of the J16 Jurkat cell line with cephalostatin 1 leads to activation of caspase-4 and caspase-2 as well as release of Smac (second mitochondria-derived activator of caspase) from the mitochondria. [12] This specific sequence of events is rarely observed in small molecule-induced apoptosis, suggesting that studying the mechanism of cephalostatin 1 may uncover new apoptotic signaling mechanisms. Therefore, cephalostatin 1 may be useful in treating tumors that have become resistant to conventional cancer therapeutics.

It was shown that cephalostatin 1 also led to formation of the multi-protein complex known as the PIDDosome (comprising proteins PIDD, RAIDD, and caspase-2). [9,10] The histone deacetylase inhibitor trichostatin A is the only other small molecule known to induce formation of the PIDDosome. [13] Cephalostatin 1 was also shown to induce an ER stress response. [11] The highly similar structures of ritterazine B and cephalostatin 1 and their nearly identical COMPARE Pearson correlation coefficient (p=0.93) suggest the compounds likely share a cellular target(s).

A few synthetically produced cephalostatin analogues, synthetic methods for preparing the cephalostatin analogues, pharmaceutical compositions containing the analogues and methods for using the analogues as active agent in pharmaceutical uses, are described in U.S. Pat. No. 5,708,164 (Winterfeldt, E. et al.); which is hereby incorportated by reference in its entirty, such as for the cephalostatin analogs, and their preparation, as described therein.

One aspect of the invention relates to a compound represented by formula I:

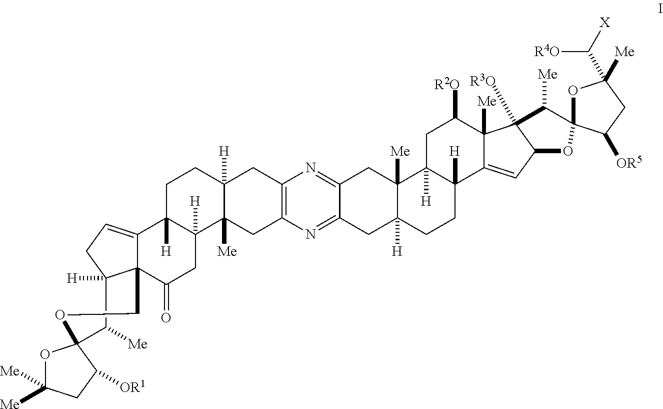

or a pharmaceutically acceptable salt, biologically active metabolite, solvate, hydrate, prodrug, enantiomer or stereoisomer thereof, wherein, independently for each occurrence,

R¹ is hydrogen, silyl, aralkyl, alkylcarboxy, arylcarboxy, heteroarylcarboxy, aralkylcarboxy or heteroaralkylcarboxy;

R² is hydrogen, silyl, aralkyl, alkylcarboxy, arylcarboxy, heteroarylcarboxy, aralkylcarboxy or heteroaralkylcarboxy;

R³ is hydrogen, silyl, aralkyl, alkylcarboxy, arylcarboxy, heteroarylcarboxy, aralkylcarboxy or heteroaralkylcarboxy;

R⁴ is hydrogen, silyl, aralkyl, alkylcarboxy, arylcarboxy, heteroarylcarboxy, aralkylcarboxy or heteroaralkylcarboxy;

R⁵ is hydrogen, silyl, aralkyl, alkylcarboxy, arylcarboxy, heteroarylcarboxy, aralkylcarboxy or heteroaralkylcarboxy; and

X is hydrogen, deuterium, tritium, alkyl, aralkyl or heteroaralkyl.

In certain embodiments, the present invention relates to any one of the above-referenced compounds, wherein R¹ is hydrogen. In certain embodiments, the present invention relates to any one of the above-referenced compounds, wherein R¹ is —C(═O)CH₃. In certain embodiments, the present invention relates to any one of the above-referenced compounds, wherein R¹ is silyl.

In certain embodiments, the present invention relates to any one of the above-referenced compounds, wherein R² is hydrogen. In certain embodiments, the present invention relates to any one of the above-referenced compounds, wherein R² is —C(═O)CH₃. In certain embodiments, the present invention relates to any one of the above-referenced compounds, wherein R² is silyl.

In certain embodiments, the present invention relates to any one of the above-referenced compounds, wherein R³ is hydrogen. In certain embodiments, the present invention relates to any one of the above-referenced compounds, wherein R¹ is —C(═O)CH₃. In certain embodiments, the present invention relates to any one of the above-referenced compounds, wherein R³ is silyl.

In certain embodiments, the present invention relates to any one of the above-referenced compounds, wherein R⁴ is hydrogen. In certain embodiments, the present invention relates to any one of the above-referenced compounds, wherein R⁴ is —C(═O)CH₃. In certain embodiments, the present invention relates to any one of the above-referenced compounds, wherein R⁴ is silyl.

In certain embodiments, the present invention relates to any one of the above-referenced compounds, wherein R⁵ is hydrogen. In certain embodiments, the present invention relates to any one of the above-referenced compounds, wherein R⁵ is —C(═O)CH₃. In certain embodiments, the present invention relates to any one of the above-referenced compounds, wherein R⁵ is silyl.

In certain embodiments, the present invention relates to any one of the above-referenced compounds, wherein X is hydrogen. In certain embodiments, the present invention relates to any one of the above-referenced compounds, wherein X is tritium. In certain embodiments, the present invention relates to any one of the above-referenced compounds, wherein X is alkyl.

In certain embodiments, the present invention relates to any one of the above-referenced compounds, wherein R¹ is hydrogen; R² is hydrogen; R³ is hydrogen; R⁴ is hydrogen; and R⁵ is hydrogen.

In certain embodiments, the present invention relates to any one of the above-referenced compounds, wherein R¹ is hydrogen; R² is hydrogen; R³ is hydrogen; R⁴ is hydrogen; R⁵ is hydrogen; and X is hydrogen.

In certain embodiments, the present invention relates to any one of the above-referenced compounds, wherein R¹ is hydrogen; R² is hydrogen; R³ is hydrogen; R⁴ is hydrogen; R⁵ is hydrogen; and X is tritium.

One aspect of the invention relates to a compound represented by formula II:

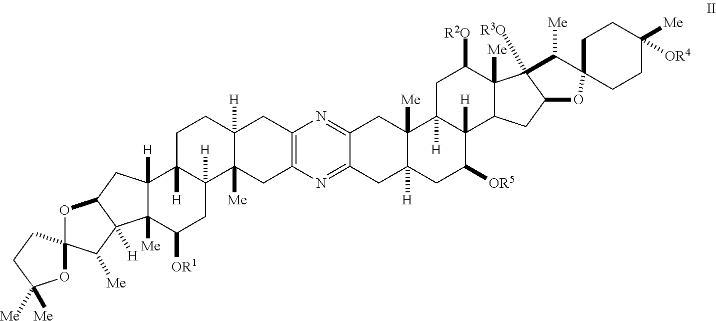

or a pharmaceutically acceptable salt, biologically active metabolite, solvate, hydrate, prodrug, enantiomer or stereoisomer thereof, wherein, independently for each occurrence,

R¹ is hydrogen, silyl, aralkyl, alkylcarboxy, arylcarboxy, heteroarylcarboxy, aralkylcarboxy or heteroaralkylcarboxy;

R² is hydrogen, silyl, aralkyl, alkylcarboxy, arylcarboxy, heteroarylcarboxy, aralkylcarboxy or heteroaralkylcarboxy;

R³ is hydrogen, silyl, aralkyl, alkylcarboxy, arylcarboxy, heteroarylcarboxy, aralkylcarboxy or heteroaralkylcarboxy;

R⁴ is hydrogen, silyl, aralkyl, alkylcarboxy, arylcarboxy, heteroarylcarboxy, aralkylcarboxy or heteroaralkylcarboxy; and

R⁵ is hydrogen, silyl, aralkyl, alkylcarboxy, arylcarboxy, heteroarylcarboxy, aralkylcarboxy or heteroaralkylcarboxy.

In certain embodiments, the present invention relates to any one of the above-referenced compounds, wherein R¹ is hydrogen. In certain embodiments, the present invention relates to any one of the above-referenced compounds, wherein R¹ is —C(═O)CH₃. In certain embodiments, the present invention relates to any one of the above-referenced compounds, wherein R¹ is silyl.

In certain embodiments, the present invention relates to any one of the above-referenced compounds, wherein R² is hydrogen. In certain embodiments, the present invention relates to any one of the above-referenced compounds, wherein R² is —C(═O)CH₃. In certain embodiments, the present invention relates to any one of the above-referenced compounds, wherein R² is silyl.

In certain embodiments, the present invention relates to any one of the above-referenced compounds, wherein R³ is hydrogen. In certain embodiments, the present invention relates to any one of the above-referenced compounds, wherein R¹ is —C(═O)CH₃. In certain embodiments, the present invention relates to any one of the above-referenced compounds, wherein R³ is silyl.

In certain embodiments, the present invention relates to any one of the above-referenced compounds, wherein R⁴ is hydrogen. In certain embodiments, the present invention relates to any one of the above-referenced compounds, wherein R⁴ is —C(═O)CH₃. In certain embodiments, the present invention relates to any one of the above-referenced compounds, wherein R⁴ is silyl.

In certain embodiments, the present invention relates to any one of the above-referenced compounds, wherein R⁵ is hydrogen. In certain embodiments, the present invention relates to any one of the above-referenced compounds, wherein R⁵ is —C(═O)CH₃. In certain embodiments, the present invention relates to any one of the above-referenced compounds, wherein R⁵ is silyl.

In certain embodiments, the present invention relates to any one of the above-referenced compounds, wherein R¹ is hydrogen; R² is hydrogen; R³ is hydrogen; R⁴ is hydrogen; and R⁵ is hydrogen.

### OSW-1

OSW-1 (2) is more abundant than cephalostatin 1, which has enabled some in vivo studies. [3] The GI₅₀ of OSW-1, taken as an average against the NCI-60, is 0.78 nM. In vivo studies of 2 have been limited, but nude mice subcutaneously implanted with P388 tumor cells demonstrated a 59% increased life span upon a single i.p. treatment of 0.01 mg/kg OSW-1. [3] OSW-1 also shows selectivity for malignant cell types over non-malignant cells. OSW-1 is 40 times more active in leukemia cells (HL-60, GI₅₀=0.04 nM) versus normal lymphocytes (GI₅₀=1.73 nM), [14] and in malignant brain tumor cells, the selectivity increases to 150 fold (U87-MG, GI₅₀=0.047 nM versus normal astrocytes GI₅₀=7.13 nM). [14] Finally, CLL (chronic lymphocytic leukemia) cells taken from patients that were refractory to fludarabine, a commonly used chemotherapeutic for treatment of CLL and other blood cancers, were highly sensitive to OSW-1 (GI₅₀=0.3 nM). [14] These studies, albeit limited, demonstrate that OSW-1 is quite selective at inhibiting the growth of some tumors in vivo.

The use of OSW-1, and analogs thereof, for treating pancreatic cancers, leukemias, colon cancers, malignant gliomas and other brain tumors, and ovarian cancers is disclosed in US Patent Application Publication No. 20050004044 (Huang, P. et al.); which is hereby incorporated by reference in its entirty, such as for the preparation of OSW-1 analogs.

One aspect of the invention relates to a compound represented by formula III:

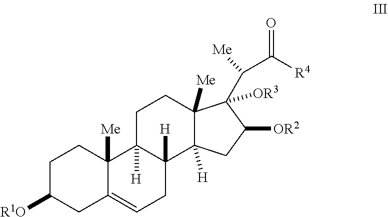

or a pharmaceutically acceptable salt, biologically active metabolite, solvate, hydrate, prodrug, enantiomer or stereoisomer thereof, wherein, independently for each occurrence,

R¹ is hydrogen, silyl, aralkyl, alkylcarboxy, arylcarboxy, heteroarylcarboxy, aralkylcarboxy or heteroaralkylcarboxy;

R² is hydrogen or a carbohydrate;

R³ is hydrogen, silyl, aralkyl, alkylcarboxy, arylcarboxy, heteroarylcarboxy, aralkylcarboxy or heteroaralkylcarboxy; and

R⁴ is alkyl, aralkyl or heteroaralkyl.

In certain embodiments, the present invention relates to any one of the above-referenced compounds, wherein R¹ is hydrogen. In certain embodiments, the present invention relates to any one of the above-referenced compounds, wherein R¹ is —C(═O)CH₃. In certain embodiments, the present invention relates to any one of the above-referenced compounds, wherein R¹ is silyl.

In certain embodiments, the present invention relates to any one of the above-referenced compounds, wherein R² is hydrogen. In certain embodiments, the present invention relates to any one of the above-referenced compounds, wherein R² is a carbohydrate. In certain embodiments, the present invention relates to any one of the above-referenced compounds, wherein R² is a monosaccharide, disaccharide or trisaccharide. In certain embodiments, the present invention relates to any one of the above-referenced compounds, wherein R² is a disaccharide. In certain embodiments, the present invention relates to any one of the above-referenced compounds, wherein R² is

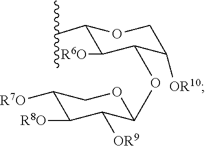

wherein R⁶, R⁷, R⁸, R⁹ and R¹⁰ are independently selected from the group consisting of hydrogen, silyl, aralkyl, alkylcarboxy, arylcarboxy, heteroarylcarboxy, aralkylcarboxy and heteroaralkylcarboxy.

In certain embodiments, the present invention relates to any one of the above-referenced compounds, wherein R³ is hydrogen. In certain embodiments, the present invention relates to any one of the above-referenced compounds, wherein R¹ is —C(═O)CH₃. In certain embodiments, the present invention relates to any one of the above-referenced compounds, wherein R³ is silyl.

In certain embodiments, the present invention relates to any one of the above-referenced compounds, wherein R⁴ is alkyl. In certain embodiments, the present invention relates to any one of the above-referenced compounds, wherein R⁴ is —CH₂CH₂CH(CH₃)₂.

In certain embodiments, the present invention relates to any one of the above-referenced compounds, wherein R⁶ is hydrogen. In certain embodiments, the present invention relates to any one of the above-referenced compounds, wherein R⁶ is —C(═O)CH₃. In certain embodiments, the present invention relates to any one of the above-referenced compounds, wherein R⁶ is silyl.

In certain embodiments, the present invention relates to any one of the above-referenced compounds, wherein R⁷ is hydrogen. In certain embodiments, the present invention relates to any one of the above-referenced compounds, wherein R⁷ is —C(═O)CH₃. In certain embodiments, the present invention relates to any one of the above-referenced compounds, wherein R⁷ is silyl.

In certain embodiments, the present invention relates to any one of the above-referenced compounds, wherein R⁸ is hydrogen. In certain embodiments, the present invention relates to any one of the above-referenced compounds, wherein R⁸ is —C(═O)CH₃. In certain embodiments, the present invention relates to any one of the above-referenced compounds, wherein R⁸ is silyl.

In certain embodiments, the present invention relates to any one of the above-referenced compounds, wherein R⁹ is hydrogen. In certain embodiments, the present invention relates to any one of the above-referenced compounds, wherein R⁹ is —C(═O)CH₃. In certain embodiments, the present invention relates to any one of the above-referenced compounds, wherein R⁹ is silyl.

In certain embodiments, the present invention relates to any one of the above-referenced compounds, wherein R¹⁰ is hydrogen. In certain embodiments, the present invention relates to any one of the above-referenced compounds, wherein R¹⁰ is —C(═O)CH₃. In certain embodiments, the present invention relates to any one of the above-referenced compounds, wherein R¹⁰ is silyl.

In certain embodiments, the present invention relates to any one of the above-referenced compounds, wherein R¹ is hydrogen; R³ is hydrogen; and R⁴ is alkyl.

In certain embodiments, the present invention relates to any one of the above-referenced compounds, wherein R¹ is hydrogen; R² is

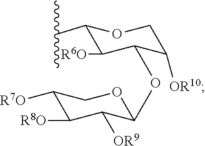

R³ is hydrogen; R⁴ is alkyl; and R⁶, R⁷, R⁸, R⁹ and R¹⁰ are independently selected from the group consisting of hydrogen, silyl, aralkyl, alkylcarboxy, arylcarboxy, heteroarylcarboxy, aralkylcarboxy and heteroaralkylcarboxy.

In certain embodiments, the present invention relates to any one of the above-referenced compounds, wherein R¹ is hydrogen; R² is

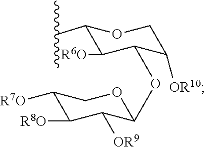

R³ is hydrogen; R⁴ is alkyl; and R⁶, R⁷, R⁸ and R¹⁰ are hydrogen; and R⁹ is alkylcarboxy, arylcarboxy, heteroarylcarboxy, aralkylcarboxy and heteroaralkylcarboxy.

### Schweinfurthins

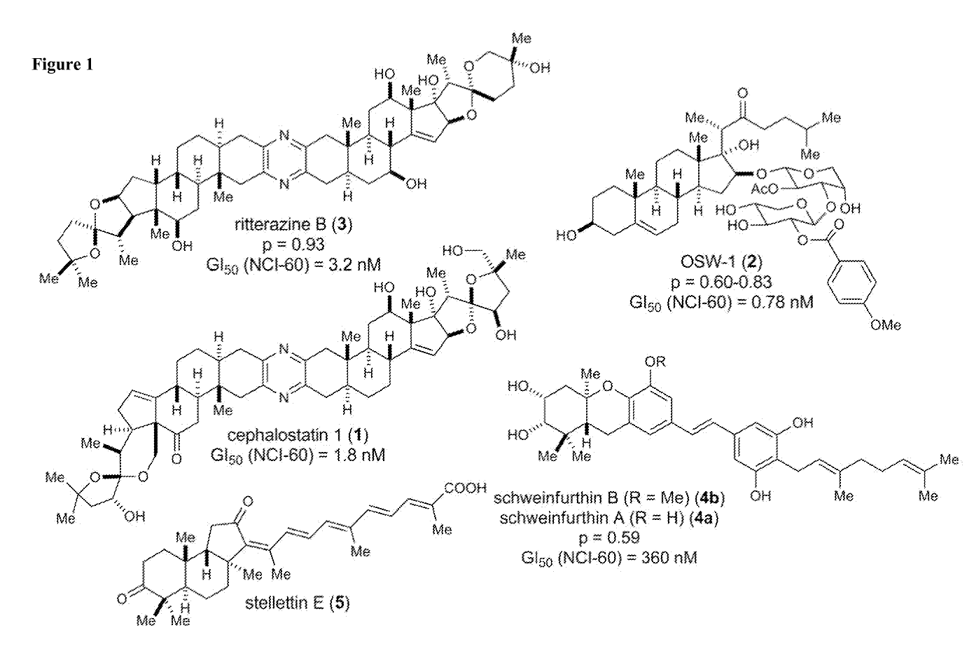

The family of natural products known as the schweinfurthins currently includes four compounds isolated from the African plant Macaranga schweinfurthii Pax (see FIG. 1). Schweinfurthin A (4) (average GI₅₀=360 nM vs. NCI-60) exhibits a similar cytotoxicity profile to cephalostatin 1 (p=0.59 with 1) [8] and the other CRAMs. Schweinfurthins B and D also display significant activity in the NCI's 60-cell-line anticancer assay with mean GI₅₀ valuss of less than 1 μM. Their biological activity has attracted interest because some CNS, renal, and breast cancer cell lines are among the types most sensitive to these compounds. Inspection of the spectrum of activity shows no correlation with any currently used agents and suggests that these compounds may be acting at a previously unrecognized target or through a novel mechanism. Prior to the inventions disclosed herein, it was not known that schweinfurthin A bound to OSBPs.

Like cephalostatin 1, only small quantities of schweinfurthin A have been available from natural sources, which has limited in vivo studies. [8] Repeated attempts to isolate larger samples of the schweinfurthins from natural sources have not been fruitful. A synthesis of schweinfurthin A has not yet been achieved, although the Wiemer group has achieved total syntheses of schweinfurthin B, E and F. [15, 16] See also, US Patent Application Publication No. 2008/0227852 to Wiemer, D. et al.; hereby incorporated by reference in its entity. For the preparation of some related stilbene derivatives, see U.S. Pat. No. 7,321,050 to Chen, G. et al.; hereby incorporated by reference in its entirety.

In a single reported mouse xenograft experiment with schweinfurthin A, i.p. administration of 9.3 mg/kg (Q2D×4) led to “reduction in tumor volume compared to vehicle-treated controls without overt toxicity.” [8]

One aspect of the invention relates to a compound represented by formula IV:

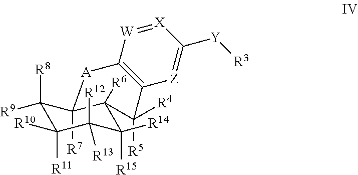

or a pharmaceutically acceptable salt, biologically active metabolite, solvate, hydrate, prodrug, enantiomer or stereoisomer thereof, wherein, independently for each occurrence,

A is —O—, —S(═O)ₙ—, —C(R^{C})₂—, or —N(R^{N})—;

n is 0, 1 or 2;

W is —C(R¹)— or —N—;

X is —C(R²)— or —N—;

Y is -alkylene-R;

Z is —C(R³)— or —N—;

R is prenylaryl or prenylheteroaryl, optionally substituted with between one and ten substituents independently selected from the group consisting of alkenyl, alkoxy, alkyl, alkylcarbonyloxy, alkylthio, alkylcarbonylthio, alkynyl, amido, amidoalkyl, amino, aminoalkyl, carboxy, cyano, formyl, halo, haloalkoxy, haloalkyl, haloalkylthio, hydroxyl, hydroxyalkyl, mercapto, mercaptoalkyl, nitro, silyl, silyloxy and silyloxyalkyl;

R¹, R¹³ and R¹¹ are independently selected from the group consisting of alkenyl, alkoxy, alkyl, alkylcarbonyloxy, alkylthio, alkylcarbonylthio, alkynyl, amido, amidoalkyl, amino, aminoalkyl, carboxy, cyano, formyl, halo, haloalkoxy, haloalkyl, haloalkylthio, hydroxyl, hydroxyalkyl, mercapto, mercaptoalkyl, nitro, silyl, silyloxy and silyloxyalkyl;

R², R³, R⁴, R⁵, R⁸, R⁹, R¹⁰, R¹² and R^{C} are independently selected from the group consisting of alkenyl, alkoxy, alkyl, alkylcarbonyloxy, alkylthio, alkylcarbonylthio, alkynyl, amido, amidoalkyl, amino, aminoalkyl, carboxy, cyano, formyl, halo, haloalkoxy, haloalkyl, haloalkylthio, hydrogen, hydroxyl, hydroxyalkyl, mercapto, mercaptoalkyl, nitro, silyl, silyloxy and silyloxyalkyl;

R⁶, R⁷, R¹⁴ and R¹⁵ are independently selected from the group consisting of hydrogen, alkyl and haloalkyl; and

R^{N} is hydrogen, alkyl, alkylcarbonyl, aralkylcarbonyl or haloalkyl.

In certain embodiments, the present invention relates to any one of the above-referenced compounds, wherein the compound is not schweinfurthin A, schweinfurthin B, schweinfurthin D, dimethyoxy-3-deoxyschweinfurthin B, 7-{2-[4-(3,7-dimethylocta-6,7-dienyl)phenyl]vinyl}-5-methoxy-1,1,4a-trimethyl-2,3,4,4a,9,9a-hexahydro-1H-xanthen-2-ol, 7-{2-[4-(3,7-dimethylocta-2,6-dienyl)-3,5-difluorophenyl]vinyl}-5-methoxy-1,1,4a-trimethyl-2,3,4,4a,9,9a-hexahydro-1H-xanthen-2-ol, 5-methoxy-1,1,4a-trimethyl-7-styryl-2,3,4,4a,9,9a-hexahydro-1H-xanthen-2-ol, 5-[2-(7-hydroxy-4-methoxy-8,8,10a-trimethyl-5,7,8,8a,9,10a-hexahydro-6H-xanthen-2-yl)vinyl]benzene-1,3-diol, 2-(8-hydroxy-3,7-dimethylocta-2,6-dienyl)-5-[2-(7-hydroxy-4-methoxy-8,8,10a-trimethyl-5,7,8,8a,9,10a-hexahydro-6H-xanthen-2-yl)vinyl]benzene-1,3-diol, or 7-[2-(3-hydroxyphenyl)vinyl]-5-methoxy-1,1,4a-trimethyl-2,3,4,4a,9,9a-hexahydro-1H-xanthen-2-ol.

In certain embodiments, the present invention relates to any one of the above-referenced compounds, wherein A is —O—.

In certain embodiments, the present invention relates to any one of the above-referenced compounds, wherein W is —C(R¹)—.

In certain embodiments, the present invention relates to any one of the above-referenced compounds, wherein R¹ is hydroxyl, alkyoxy or haloalkyloxy.

In certain embodiments, the present invention relates to any one of the above-referenced compounds, wherein R¹ is hydroxyl.

In certain embodiments, the present invention relates to any one of the above-referenced compounds, wherein X is —C(R²)—.

In certain embodiments, the present invention relates to any one of the above-referenced compounds, wherein R² is hydrogen.

In certain embodiments, the present invention relates to any one of the above-referenced compounds, wherein Y is —C(R^{C})₂C(R^{C})₂R, —C(R^{C})═C(R^{C})R, or —C≡CR.

In certain embodiments, the present invention relates to any one of the above-referenced compounds, wherein Y is trans-vinylene.

In certain embodiments, the present invention relates to any one of the above-referenced compounds, wherein R is

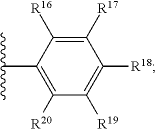

wherein R¹⁶, R¹⁷, R¹⁸, R¹⁹ and R²⁰ are independently selected from the group consisting of hydrogen, prenyl, alkenyl, alkoxy, alkyl, alkylcarbonyloxy, alkylthio, alkylcarbonylthio, alkynyl, amido, amidoalkyl, amino, aminoalkyl, carboxy, cyano, formyl, halo, haloalkoxy, haloalkyl, haloalkylthio, hydroxyl, hydroxyalkyl, mercapto, mercaptoalkyl, nitro, silyl, silyloxy and silyloxyalkyl; provided at least one R¹⁶, R¹⁷, R¹⁸, R¹⁹ and R²⁰ is prenyl.

In certain embodiments, the present invention relates to any one of the above-referenced compounds, wherein R¹⁶ is hydrogen.

In certain embodiments, the present invention relates to any one of the above-referenced compounds, wherein R¹⁷ is hydrogen, alkoxy, alkylcarbonyloxy, halo, haloalkoxy, haloalkyl, hydroxyl, hydroxyalkyl, silyloxy and silyloxyalkyl. In certain embodiments, the present invention relates to any one of the above-referenced compounds, wherein R¹⁷ is alkoxy or hydroxyl. In certain embodiments, the present invention relates to any one of the above-referenced compounds, wherein R¹⁷ is hydroxyl.

In certain embodiments, the present invention relates to any one of the above-referenced compounds, wherein R¹⁸ is prenyl. In certain embodiments, the present invention relates to any one of the above-referenced compounds, wherein R¹⁸ is

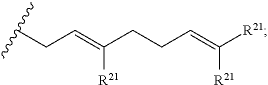

and R²¹ is hydrogen, alkyl or haloalkyl. In certain embodiments, the present invention relates to any one of the above-referenced compounds, wherein R¹⁸ is geranyl.

In certain embodiments, the present invention relates to any one of the above-referenced compounds, wherein R¹⁹ is hydrogen, alkoxy, alkylcarbonyloxy, halo, haloalkoxy, haloalkyl, hydroxyl, hydroxyalkyl, silyloxy and silyloxyalkyl. In certain embodiments, the present invention relates to any one of the above-referenced compounds, wherein R¹⁹ is alkoxy or hydroxyl. In certain embodiments, the present invention relates to any one of the above-referenced compounds, wherein R¹⁹ is hydroxyl.

In certain embodiments, the present invention relates to any one of the above-referenced compounds, wherein R²⁰ is hydrogen.

In certain embodiments, the present invention relates to any one of the above-referenced compounds, wherein Z is —C(R³)—.

In certain embodiments, the present invention relates to any one of the above-referenced compounds, wherein R³ is hydrogen.

In certain embodiments, the present invention relates to any one of the above-referenced compounds, wherein R⁴ is hydrogen, alkyl or haloalkyl. In certain embodiments, the present invention relates to any one of the above-referenced compounds, wherein R⁴ is hydrogen.

In certain embodiments, the present invention relates to any one of the above-referenced compounds, wherein R⁵ is hydrogen, alkyl or haloalkyl. In certain embodiments, the present invention relates to any one of the above-referenced compounds, wherein R⁵ is hydrogen.

In certain embodiments, the present invention relates to any one of the above-referenced compounds, wherein R⁶ is hydrogen, alkyl or haloalkyl. In certain embodiments, the present invention relates to any one of the above-referenced compounds, wherein R⁶ is hydrogen.

In certain embodiments, the present invention relates to any one of the above-referenced compounds, wherein R⁷ is hydrogen, alkyl or haloalkyl. In certain embodiments, the present invention relates to any one of the above-referenced compounds, wherein R⁷ is hydrogen.

In certain embodiments, the present invention relates to any one of the above-referenced compounds, wherein R⁸ is alkoxy, alkyl, alkylcarbonyloxy, halo, haloalkoxy, haloalkyl, hydrogen, hydroxyl, or silyloxy. In certain embodiments, the present invention relates to any one of the above-referenced compounds, wherein R⁸ is hydrogen.

In certain embodiments, the present invention relates to any one of the above-referenced compounds, wherein R⁹ is alkoxy, alkyl, alkylcarbonyloxy, halo, haloalkoxy, haloalkyl, hydrogen, hydroxyl, or silyloxy. In certain embodiments, the present invention relates to any one of the above-referenced compounds, wherein R⁹ is hydrogen.

In certain embodiments, the present invention relates to any one of the above-referenced compounds, wherein R¹⁰ alkoxy, alkyl, alkylcarbonyloxy, halo, haloalkoxy, haloalkyl, hydrogen, hydroxyl, or silyloxy. In certain embodiments, the present invention relates to any one of the above-referenced compounds, wherein R¹⁰ is hydrogen.

In certain embodiments, the present invention relates to any one of the above-referenced compounds, wherein R¹¹ is alkoxy, alkyl, alkylcarbonyloxy, halo, haloalkoxy, haloalkyl, hydroxyl, or silyloxy. In certain embodiments, the present invention relates to any one of the above-referenced compounds, wherein R¹¹ is hydroxyl.

In certain embodiments, the present invention relates to any one of the above-referenced compounds, wherein R¹² is alkoxy, alkyl, alkylcarbonyloxy, halo, haloalkoxy, haloalkyl, hydrogen, hydroxyl, or silyloxy. In certain embodiments, the present invention relates to any one of the above-referenced compounds, wherein R¹² is hydrogen.

In certain embodiments, the present invention relates to any one of the above-referenced compounds, wherein R¹³ alkoxy, alkyl, alkylcarbonyloxy, halo, haloalkoxy, haloalkyl, hydroxyl, or silyloxy. In certain embodiments, the present invention relates to any one of the above-referenced compounds, wherein R¹³ is hydroxyl.

In certain embodiments, the present invention relates to any one of the above-referenced compounds, wherein R¹⁴ is hydrogen, alkyl or haloalkyl. In certain embodiments, the present invention relates to any one of the above-referenced compounds, wherein R¹⁴ is hydrogen.

In certain embodiments, the present invention relates to any one of the above-referenced compounds, wherein R¹⁵ is hydrogen, alkyl or haloalkyl. In certain embodiments, the present invention relates to any one of the above-referenced compounds, wherein R¹⁵ is hydrogen.

### (−)-Stellettin E

(−)-Stellettin E (5) is a marine natural product also reported to have a cytotoxicity pattern similar to cephalostatin 1 (p not reported). [6] (−)-Stellettin E is a light sensitive compound only isolated in small quantities, prohibiting detailed cell-based studies and in vivo experiments. [6] However, the cellular target of (−)-stellettin E (and by analogy CRAMs) is made even more intriguing by the discovery that the compound is 117 times more cytotoxic to a HCT-116 colon cancer cell line engineered not to express the p21-CDKN1A (herein “p21”) tumor suppressor, compared to the parental HCT-116 cell line (GI₅₀=39 nM in HCT-116 p21^{-/-} versus GI₅₀=4.57 μM in HCT-116 p21^{+/+}). [6] These results suggest that (−)-stellettin E (and possibly all CRAMs) may be synthetic lethal with p21. At the outset of our project, no other CRAMs had been reported to be synthetic lethal with p21. The p21 protein, a cyclin-dependent kinase inhibitor, is often not expressed in advanced tumors, [17, 18] indicating that CRAMs, if truly p21 synthetic lethal compounds, may have an excellent therapeutic window in patients with late-stage, difficult-to-treat tumors. Since very few small molecules have been reported to be highly selective towards isogenic cell lines lacking p21, [19] this is another indication that CRAMs are compounds with interesting cellular targets and mechanisms that could impact the study and treatment of cancer.

One aspect of the invention relates to a compound represented by formula V:

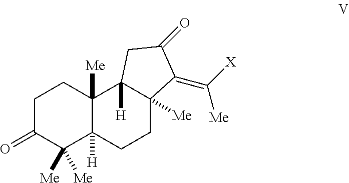

or a pharmaceutically acceptable salt, biologically active metabolite, solvate, hydrate, prodrug, enantiomer or stereoisomer thereof, wherein, independently for each occurrence,

X is

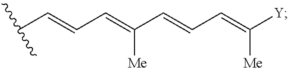

Y is —CH₂OR¹, —C(═O)R², —C(═O)OR³ or —C(═O)NHR³; and

R¹ is hydrogen, silyl, aralkyl, alkylcarboxy, arylcarboxy, heteroarylcarboxy, aralkylcarboxy or heteroaralkylcarboxy;

R² is hydrogen, alkyl, aryl, heteroaryl, aralkyl or heteroaralkyl; and

R³ is hydrogen, alkyl, aryl, heteroaryl, aralkyl, heteroaralkyl, alkylcarboxy, arylcarboxy, heteroarylcarboxy, aralkylcarboxy or heteroaralkylcarboxy.

In certain embodiments, the present invention relates to any one of the above-referenced compounds, wherein Y is —CH₂OR¹. In certain embodiments, the present invention relates to any one of the above-referenced compounds, wherein Y is —C(═O)R². In certain embodiments, the present invention relates to any one of the above-referenced compounds, wherein Y is —C(═O)OR³. In certain embodiments, the present invention relates to any one of the above-referenced compounds, wherein Y is —C(═O)NHR³.

In certain embodiments, the present invention relates to any one of the above-referenced compounds, wherein R¹ is hydrogen. In certain embodiments, the present invention relates to any one of the above-referenced compounds, wherein R¹ is —C(═O)CH₃. In certain embodiments, the present invention relates to any one of the above-referenced compounds, wherein R¹ is silyl.

In certain embodiments, the present invention relates to any one of the above-referenced compounds, wherein R² is alkyl. In certain embodiments, the present invention relates to any one of the above-referenced compounds, wherein R² is aralkyl or heteroaralkyl.

In certain embodiments, the present invention relates to any one of the above-referenced compounds, wherein R³ is hydrogen. In certain embodiments, the present invention relates to any one of the above-referenced compounds, wherein R³ is alkyl, aryl, heteroaryl, aralkyl, heteroaralkyl, alkylcarboxy, arylcarboxy, heteroarylcarboxy, aralkylcarboxy or heteroaralkylcarboxy.

In certain embodiments, the present invention relates to any one of the above-referenced compounds, wherein Y is —C(═O)OR³; and R³ is hydrogen.

### Pharmaceutical Compositions

One or more compounds of the invention (e.g., a compound of formula I, II, III, IV or V), can be administered alone to a human patient or in the form of pharmaceutical compositions where they are mixed with biologically suitable carriers or excipient(s) at doses to treat or ameliorate a disease or condition as described herein. Mixtures of these compounds can also be administered to the patient as a simple mixture or in suitable formulated pharmaceutical compositions. A therapeutically effective dose refers to that amount of the compound or compounds sufficient to result in the prevention or attenuation of a disease or condition as described herein. Techniques for formulation and administration of the compounds of the instant application may be found in references well known to one of ordinary skill in the art, such as “Remington's Pharmaceutical Sciences,” Mack Publishing Co., Easton, Pa., latest edition.

Suitable routes of administration include, for example, oral, eyedrop, rectal, transmucosal, topical, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one may administer the compound in a local rather than a systemic manner, for example, via injection of the compound directly into an edematous site, often in a depot or sustained release formulation.

Furthermore, one may administer the drug in a targeted drug delivery system, for example, in a liposome coated with endothelial cell-specific antibody.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof, such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds can be formulated for parenteral administration by injection, e.g., bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly or by intramuscular injection). Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

An example of a pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethysulfoxide also may be employed, although usually at the cost of greater toxicity. Additionally, the compounds may be delivered using a sustained-release system, such as semi-permeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions (i.e., pharmaceutically acceptable salts). A “pharmaceutically acceptable salt” means any non-toxic salt that, upon administration to a recipient, is capable of providing, either directly or indirectly, a compound or a prodrug of a compound of this invention. A “pharmaceutically acceptable counterion” is an ionic portion of a salt that is not toxic when released from the salt upon administration to a recipient. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

Acids commonly employed to form pharmaceutically acceptable salts include inorganic acids such as hydrogen bisulfide, hydrochloric, hydrobromic, hydroiodic, sulfuric and phosphoric acid, as well as organic acids such as para-toluenesulfonic, salicylic, tartaric, bitartaric, ascorbic, maleic, besylic, fumaric, gluconic, glucuronic, formic, glutamic, methanesulfonic, ethanesulfonic, benzenesulfonic, lactic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and the like salts. Preferred pharmaceutically acceptable acid addition salts include those formed with mineral acids such as hydrochloric acid and hydrobromic acid, and especially those formed with organic acids such as maleic acid.

Suitable bases for forming pharmaceutically acceptable salts with acidic functional groups include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl, N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art.

### Dosage

For any compound used in a method of the present invention, the therapeutically effective dose can be estimated initially from cellular assays. For example, a dose can be formulated in cellular and animal models to achieve a circulating concentration range that includes the IC₅₀ as determined in cellular assays (i.e., the concentration of the test compound which achieves a half-maximal inhibition). In some cases it is appropriate to determine the IC₅₀ in the presence of 3 to 5% serum albumin since such a determination approximates the binding effects of plasma protein on the compound. Such information can be used to more accurately determine useful doses in humans.

A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the maximum tolerated dose (MTD) and the ED₅₀ (effective dose for 50% maximal response). The dose ratio between toxic and therapeutic effects is the therapeutic index and it can be expressed as the ratio between MTD and ED₅₀. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p 1). In the treatment of crises, the administration of an acute bolus or an infusion approaching the MTD may be required to obtain a rapid response.

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g., the concentration necessary to achieve 50-90% inhibition of protein kinase using the assays described herein. Dosages necessary to achieve the MEC will depend on individual characteristics and route of administration. However, HPLC assays or bioassays can be used to determine plasma concentrations.

Dosage intervals can also be determined using the MEC value. Compounds should be administered using a regimen which maintains plasma levels above the MEC for 10-90% of the time, preferably between 30-90% and most preferably between 50-90% until the desired amelioration of symptoms is achieved. In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

### Exemplary Formulations

In some formulations it may be beneficial to use the compounds of the present invention in the form of particles of very small size, for example as obtained by fluid energy milling.

The use of compounds of the present invention in the manufacture of pharmaceutical compositions is illustrated by the following description. In this description the term “active compound” denotes any compound of the invention.

Capsules containing an active compound can be prepared. In the preparation of capsules, 10 parts by weight of active compound and 240 parts by weight of lactose can be de-aggregated and blended. The mixture can be filled into hard gelatin capsules, each capsule containing a unit dose or part of a unit dose of active compound.

Tablets can be prepared, for example, from the active compound (10 parts by weight), lactose (190 parts by weight), maize starch (22 parts by weight), polyvinylpyrrolidone (10 parts by weight) and magnesium sterate (3 parts by weight). The active compound, the lactose and some of the starch can be de-aggregated, blended and the resulting mixture can be granulated with a solution of the polyvinylpyrrolidone in ethanol. The dry granulate can be blended with the magnesium stearate and the rest of the starch. The mixture is then compressed in a tabletting machine to give tablets each containing a unit dose or a part of a unit dose of active compound.

The tablets can be enteric coated in a conventional manner using a solution of 20% cellulose acetate phthalate and 3% diethyl phthalate in ethanol:dichloromethane (1:1).

Suppositories containing an active compound can be prepared. In the preparation of suppositories, for example, 100 parts by weight of active compound can be incorporated in 1300 parts by weight of triglyceride suppository base and the mixture formed into suppositories each containing a therapeutically effective amount of active ingredient.

### Immobilized Compounds and Methods of Screening

One aspect of the invention relates to an immobilized compound, comprising a solid support, a linker, and a compound selected from the above-referenced compounds, such as OSW-1; wherein said compound is bound to said solid support via the linker.

The linker is a functional chemical moiety attaching compound to solid support or soluble support which can be cleaved to release compounds from the support. Careful choice of linker allows cleavage to be performed under appropriate conditions compatible with the stability of the compound and assay method. In certain embodiments, the present invention relates to the above-referenced immobilized compound, wherein the compound is bound to the linker through an oxygen atom of the compound, such as a hydroxyl. In certain embodiments, the present invention relates to the above-referenced immobilized compound, wherein the linker is a diradical formed from a dicarboxylic acid, a diester or a diamide. In certain embodiments, the present invention relates to the above-referenced immobilized compound, wherein the linker is —C(═O)CH₂N(H)C₆H₄CH₂N(H)C(═O)—.

The solid support is a soluble, functionalized, polymeric material to which a compound may be attached (via a linker) allowing the compound to be readily separated (by filtration, centrifugation, etc.) and/or detected. In certain embodiments, the present invention relates to the above-referenced immobilized compound, wherein the solid support is selected from the group consisting of inorganic powders such as silica, magnesium sulfate, and alumina; natural polymeric materials, particularly cellulosic materials and materials derived from cellulose, such as fiber containing papers, e.g., filter paper, chromatographic paper, etc.; synthetic or modified naturally occurring polymers, such as nitrocellulose, cellulose acetate, poly (vinyl chloride), polyacrylamide, cross linked dextran, agarose, polyacrylate, polyethylene, polypropylene, poly(4-methylbutene), polystyrene, polymethacrylate, poly(ethylene terephthalate), nylon, poly(vinyl butyrate), etc.; either used by themselves or in conjunction with other materials; glass available as Bioglass, ceramics, metals, and the like.

Immobilized compounds can be used to screen for compounds which can bind oxysterol binding proteins. One aspect of the invention relates to a method for determining if a compound is an oxysterol binding comprising the steps of asscoicating an immobilized compound with an oxysterol binding protein to form a complex; contacting the complex with a compound; and measuring the extent of the disruption of the complex. Screening assasys are discussed in more detail in the Exemplification.

### Exemplary Methods of Use

Certain aspects of the invention relate to a method of treating an oxysterol binding protein-related disease or condition, comprising the step of administering a therapeutically effective amount of a compound of formula I to a subject in need thereof; wherein the compound of formula I is represented by

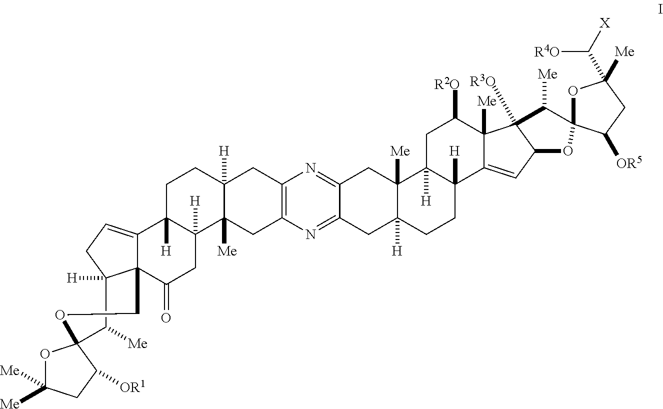

or a pharmaceutically acceptable salt, biologically active metabolite, solvate, hydrate, prodrug, enantiomer or stereoisomer thereof, wherein, independently for each occurrence,

R¹ is hydrogen, silyl, aralkyl, alkylcarboxy, arylcarboxy, heteroarylcarboxy, aralkylcarboxy or heteroaralkylcarboxy;

R² is hydrogen, silyl, aralkyl, alkylcarboxy, arylcarboxy, heteroarylcarboxy, aralkylcarboxy or heteroaralkylcarboxy;

R³ is hydrogen, silyl, aralkyl, alkylcarboxy, arylcarboxy, heteroarylcarboxy, aralkylcarboxy or heteroaralkylcarboxy;

R⁴ is hydrogen, silyl, aralkyl, alkylcarboxy, arylcarboxy, heteroarylcarboxy, aralkylcarboxy or heteroaralkylcarboxy;

R⁵ is hydrogen, silyl, aralkyl, alkylcarboxy, arylcarboxy, heteroarylcarboxy, aralkylcarboxy or heteroaralkylcarboxy; and

X is hydrogen, deuterium, tritium, alkyl, aralkyl or heteroaralkyl.

In certain embodiments, the present invention relates to any one of the above-referenced methods, wherein R¹ is hydrogen. In certain embodiments, the present invention relates to any one of the above-referenced methods, wherein R¹ is —C(═O)CH₃. In certain embodiments, the present invention relates to any one of the above-referenced methods, wherein R¹ is silyl.

In certain embodiments, the present invention relates to any one of the above-referenced methods, wherein R² is hydrogen. In certain embodiments, the present invention relates to any one of the above-referenced methods, wherein R² is —C(═O)CH₃. In certain embodiments, the present invention relates to any one of the above-referenced methods, wherein R² is silyl.

In certain embodiments, the present invention relates to any one of the above-referenced methods, wherein R³ is hydrogen. In certain embodiments, the present invention relates to any one of the above-referenced methods, wherein R¹ is —C(═O)CH₃. In certain embodiments, the present invention relates to any one of the above-referenced methods, wherein R³ is silyl.

In certain embodiments, the present invention relates to any one of the above-referenced methods, wherein R⁴ is hydrogen. In certain embodiments, the present invention relates to any one of the above-referenced methods, wherein R⁴ is —C(═O)CH₃. In certain embodiments, the present invention relates to any one of the above-referenced methods, wherein R⁴ is silyl.

In certain embodiments, the present invention relates to any one of the above-referenced methods, wherein R⁵ is hydrogen. In certain embodiments, the present invention relates to any one of the above-referenced methods, wherein R⁵ is —C(═O)CH₃. In certain embodiments, the present invention relates to any one of the above-referenced methods, wherein R⁵ is silyl.

In certain embodiments, the present invention relates to any one of the above-referenced methods, wherein X is hydrogen. In certain embodiments, the present invention relates to any one of the above-referenced methods, wherein X is tritium. In certain embodiments, the present invention relates to any one of the above-referenced methods, wherein X is alkyl.

In certain embodiments, the present invention relates to any one of the above-referenced methods, wherein R¹ is hydrogen; R² is hydrogen; R³ is hydrogen; R⁴ is hydrogen; and R⁵ is hydrogen.

In certain embodiments, the present invention relates to any one of the above-referenced methods, wherein R¹ is hydrogen; R² is hydrogen; R³ is hydrogen; R⁴ is hydrogen; R⁵ is hydrogen; and X is hydrogen.

In certain embodiments, the present invention relates to any one of the above-referenced methods, wherein R¹ is hydrogen; R² is hydrogen; R³ is hydrogen; R⁴ is hydrogen; R⁵ is hydrogen; and X is tritium.

Certain aspects of the invention relate to a method of treating an oxysterol binding protein-related disease or condition, comprising the step of administering a therapeutically effective amount of a compound of formula II to a subject in need thereof; wherein the compound of formula II is represented by

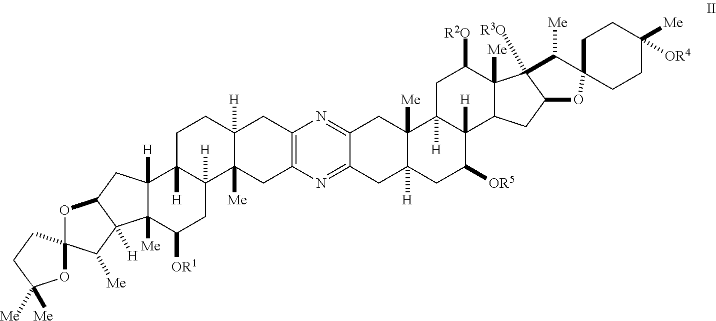

or a pharmaceutically acceptable salt, biologically active metabolite, solvate, hydrate, prodrug, enantiomer or stereoisomer thereof, wherein, independently for each occurrence,

R¹ is hydrogen, silyl, aralkyl, alkylcarboxy, arylcarboxy, heteroarylcarboxy, aralkylcarboxy or heteroaralkylcarboxy;

R² is hydrogen, silyl, aralkyl, alkylcarboxy, arylcarboxy, heteroarylcarboxy, aralkylcarboxy or heteroaralkylcarboxy;

R³ is hydrogen, silyl, aralkyl, alkylcarboxy, arylcarboxy, heteroarylcarboxy, aralkylcarboxy or heteroaralkylcarboxy;

R⁴ is hydrogen, silyl, aralkyl, alkylcarboxy, arylcarboxy, heteroarylcarboxy, aralkylcarboxy or heteroaralkylcarboxy; and

R⁵ is hydrogen, silyl, aralkyl, alkylcarboxy, arylcarboxy, heteroarylcarboxy, aralkylcarboxy or heteroaralkylcarboxy.

In certain embodiments, the present invention relates to any one of the above-referenced methods, wherein R¹ is hydrogen. In certain embodiments, the present invention relates to any one of the above-referenced methods, wherein R¹ is —C(═O)CH₃. In certain embodiments, the present invention relates to any one of the above-referenced methods, wherein R¹ is silyl.

In certain embodiments, the present invention relates to any one of the above-referenced methods, wherein R² is hydrogen. In certain embodiments, the present invention relates to any one of the above-referenced methods, wherein R² is —C(═O)CH₃. In certain embodiments, the present invention relates to any one of the above-referenced methods, wherein R² is silyl.

In certain embodiments, the present invention relates to any one of the above-referenced methods, wherein R³ is hydrogen. In certain embodiments, the present invention relates to any one of the above-referenced methods, wherein R¹ is —C(═O)CH₃. In certain embodiments, the present invention relates to any one of the above-referenced methods, wherein R³ is silyl.

In certain embodiments, the present invention relates to any one of the above-referenced methods, wherein R⁴ is hydrogen. In certain embodiments, the present invention relates to any one of the above-referenced methods, wherein R⁴ is —C(═O)CH₃. In certain embodiments, the present invention relates to any one of the above-referenced methods, wherein R⁴ is silyl.

In certain embodiments, the present invention relates to any one of the above-referenced methods, wherein R⁵ is hydrogen. In certain embodiments, the present invention relates to any one of the above-referenced methods, wherein R⁵ is —C(═O)CH₃. In certain embodiments, the present invention relates to any one of the above-referenced methods, wherein R⁵ is silyl.

In certain embodiments, the present invention relates to any one of the above-referenced methods, wherein R¹ is hydrogen; R² is hydrogen; R³ is hydrogen; R⁴ is hydrogen; and R⁵ is hydrogen.

Certain aspects of the invention relate to a method of treating an oxysterol binding protein-related disease or condition, comprising the step of administering a therapeutically effective amount of a compound of formula III to a subject in need thereof; wherein the compound of formula III is represented by

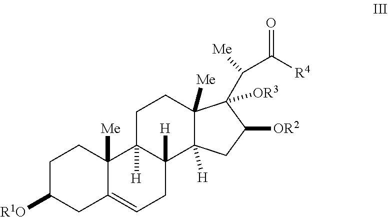

or a pharmaceutically acceptable salt, biologically active metabolite, solvate, hydrate, prodrug, enantiomer or stereoisomer thereof, wherein, independently for each occurrence,

R¹ is hydrogen, silyl, aralkyl, alkylcarboxy, arylcarboxy, heteroarylcarboxy, aralkylcarboxy or heteroaralkylcarboxy;

R² is hydrogen or a carbohydrate;

R³ is hydrogen, silyl, aralkyl, alkylcarboxy, arylcarboxy, heteroarylcarboxy, aralkylcarboxy or heteroaralkylcarboxy; and

R⁴ is alkyl, aralkyl or heteroaralkyl.

In certain embodiments, the present invention relates to any one of the above-referenced methods, wherein R¹ is hydrogen. In certain embodiments, the present invention relates to any one of the above-referenced methods, wherein R¹ is —C(═O)CH₃. In certain embodiments, the present invention relates to any one of the above-referenced methods, wherein R¹ is silyl.

In certain embodiments, the present invention relates to any one of the above-referenced methods, wherein R² is hydrogen. In certain embodiments, the present invention relates to any one of the above-referenced methods, wherein R² is a carbohydrate. In certain embodiments, the present invention relates to any one of the above-referenced methods, wherein R² is a monosaccharide, disaccharide or trisaccharide. In certain embodiments, the present invention relates to any one of the above-referenced methods, wherein R² is a disaccharide. In certain embodiments, the present invention relates to any one of the above-referenced methods, wherein R² is

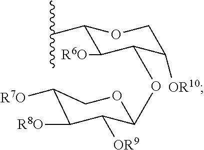

wherein R⁶, R⁷, R⁸, R⁹ and R¹⁰ are independently selected from the group consisting of hydrogen, silyl, aralkyl, alkylcarboxy, arylcarboxy, heteroarylcarboxy, aralkylcarboxy and heteroaralkylcarboxy.

In certain embodiments, the present invention relates to any one of the above-referenced methods, wherein R³ is hydrogen. In certain embodiments, the present invention relates to any one of the above-referenced methods, wherein R¹ is —C(═O)CH₃. In certain embodiments, the present invention relates to any one of the above-referenced methods, wherein R³ is silyl.

In certain embodiments, the present invention relates to any one of the above-referenced methods, wherein R⁴ is alkyl. In certain embodiments, the present invention relates to any one of the above-referenced methods, wherein R⁴ is —CH₂CH₂CH(CH₃)₂.

In certain embodiments, the present invention relates to any one of the above-referenced methods, wherein R⁶ is hydrogen. In certain embodiments, the present invention relates to any one of the above-referenced methods, wherein R⁶ is —C(═O)CH₃. In certain embodiments, the present invention relates to any one of the above-referenced methods, wherein R⁶ is silyl.

In certain embodiments, the present invention relates to any one of the above-referenced methods, wherein R⁷ is hydrogen. In certain embodiments, the present invention relates to any one of the above-referenced methods, wherein R⁷ is —C(═O)CH₃. In certain embodiments, the present invention relates to any one of the above-referenced methods, wherein R⁷ is silyl.

In certain embodiments, the present invention relates to any one of the above-referenced methods, wherein R⁸ is hydrogen. In certain embodiments, the present invention relates to any one of the above-referenced methods, wherein R⁸ is —C(═O)CH₃. In certain embodiments, the present invention relates to any one of the above-referenced methods, wherein R⁸ is silyl.

In certain embodiments, the present invention relates to any one of the above-referenced methods, wherein R⁹ is hydrogen. In certain embodiments, the present invention relates to any one of the above-referenced methods, wherein R⁹ is —C(═O)CH₃. In certain embodiments, the present invention relates to any one of the above-referenced methods, wherein R⁹ is silyl.

In certain embodiments, the present invention relates to any one of the above-referenced methods, wherein R¹⁰ is hydrogen. In certain embodiments, the present invention relates to any one of the above-referenced methods, wherein R¹⁰ is —C(═O)CH₃. In certain embodiments, the present invention relates to any one of the above-referenced methods, wherein R¹⁰ is silyl.

In certain embodiments, the present invention relates to any one of the above-referenced methods, wherein R¹ is hydrogen; R³ is hydrogen; and R⁴ is alkyl.

In certain embodiments, the present invention relates to any one of the above-referenced methods, wherein R¹ is hydrogen; R² is

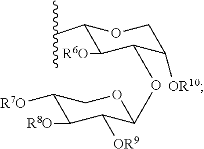

R³ is hydrogen; R⁴ is alkyl; and R⁶, R⁷, R⁸, R⁹ and R¹⁰ are independently selected from the group consisting of hydrogen, silyl, aralkyl, alkylcarboxy, arylcarboxy, heteroarylcarboxy, aralkylcarboxy and heteroaralkylcarboxy.

In certain embodiments, the present invention relates to any one of the above-referenced methods, wherein R¹ is hydrogen; R² is

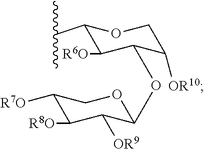

R³ is hydrogen; R⁴ is alkyl; and R⁶, R⁷, R⁸ and R¹⁰ are hydrogen; and R⁹ is alkylcarboxy, arylcarboxy, heteroarylcarboxy, aralkylcarboxy and heteroaralkylcarboxy.

Certain aspects of the invention relate to a method of treating an oxysterol binding protein-related disease or condition, comprising the step of administering a therapeutically effective amount of a compound of formula IV to a subject in need thereof; wherein the compound of formula IV is represented by:

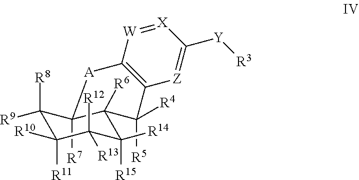

or a pharmaceutically acceptable salt, biologically active metabolite, solvate, hydrate, prodrug, enantiomer or stereoisomer thereof, wherein, independently for each occurrence,

A is —O—, —S(═O)ₙ—, —C(R^{C})₂—, or —N(R^{N})—;

n is 0, 1 or 2;

W is —C(R¹)— or —N—;

X is —C(R²)— or —N—;

Y is -alkylene-R;

Z is —C(R³)— or —N—;

R is prenylaryl or prenylheteroaryl, optionally substituted with between one and ten substituents independently selected from the group consisting of alkenyl, alkoxy, alkyl, alkylcarbonyloxy, alkylthio, alkylcarbonylthio, alkynyl, amido, amidoalkyl, amino, aminoalkyl, carboxy, cyano, formyl, halo, haloalkoxy, haloalkyl, haloalkylthio, hydroxyl, hydroxyalkyl, mercapto, mercaptoalkyl, nitro, silyl, silyloxy and silyloxyalkyl;

R¹, R¹³ and R¹¹ are independently selected from the group consisting of alkenyl, alkoxy, alkyl, alkylcarbonyloxy, alkylthio, alkylcarbonylthio, alkynyl, amido, amidoalkyl, amino, aminoalkyl, carboxy, cyano, formyl, halo, haloalkoxy, haloalkyl, haloalkylthio, hydroxyl, hydroxyalkyl, mercapto, mercaptoalkyl, nitro, silyl, silyloxy and silyloxyalkyl;

R², R³, R⁴, R⁵, R⁸, R⁹, R¹⁰, R¹² and R^{C} are independently selected from the group consisting of alkenyl, alkoxy, alkyl, alkylcarbonyloxy, alkylthio, alkylcarbonylthio, alkynyl, amido, amidoalkyl, amino, aminoalkyl, carboxy, cyano, formyl, halo, haloalkoxy, haloalkyl, haloalkylthio, hydrogen, hydroxyl, hydroxyalkyl, mercapto, mercaptoalkyl, nitro, silyl, silyloxy and silyloxyalkyl; and

R⁶, R⁷, R¹⁴ and R¹⁵ are independently selected from the group consisting of hydrogen, alkyl and haloalkyl; and

R^{N} is hydrogen, alkyl, alkylcarbonyl, aralkylcarbonyl or haloalkyl.

In certain embodiments, the present invention relates to any one of the above-referenced methods, wherein the compound is not schweinfurthin A, schweinfurthin B, schweinfurthin D, dimethyoxy-3-deoxyschweinfurthin B, 7-{2-[4-(3,7-dimethylocta-6,7-dienyl)phenyl]vinyl}-5-methoxy-1,1,4a-trimethyl-2,3,4,4a,9,9a-hexahydro-1H-xanthen-2-ol, 7-{2-[4-(3,7-dimethylocta-2,6-dienyl)-3,5-difluorophenyl]vinyl}-5-methoxy-1,1,4a-trimethyl-2,3,4,4a,9,9a-hexahydro-1H-xanthen-2-ol, 5-methoxy-1,1,4a-trimethyl-7-styryl-2,3,4,4a,9,9a-hexahydro-1H-xanthen-2-ol, 5-[2-(7-hydroxy-4-methoxy-8,8,10a-trimethyl-5,7,8,8a,9,10a-hexahydro-6H-xanthen-2-yl)vinyl]benzene-1,3-diol, 2-(8-hydroxy-3,7-dimethylocta-2,6-dienyl)-5-[2-(7-hydroxy-4-methoxy-8,8,10a-trimethyl-5,7,8,8a,9,10a-hexahydro-6H-xanthen-2-yl)vinyl]benzene-1,3-diol, or 7-[2-(3-hydroxyphenyl)vinyl]-5-methoxy-1,1,4a-trimethyl-2,3,4,4a,9,9a-hexahydro-1H-xanthen-2-ol.

In certain embodiments, the present invention relates to any one of the above-referenced methods, wherein A is —O—.

In certain embodiments, the present invention relates to any one of the above-referenced methods, wherein W is —C(R¹)—.

In certain embodiments, the present invention relates to any one of the above-referenced methods, wherein R¹ is hydroxyl, alkyoxy or haloalkyloxy.

In certain embodiments, the present invention relates to any one of the above-referenced methods, wherein R¹ is hydroxyl.

In certain embodiments, the present invention relates to any one of the above-referenced methods, wherein X is —C(R²)—.

In certain embodiments, the present invention relates to any one of the above-referenced methods, wherein R² is hydrogen.

In certain embodiments, the present invention relates to any one of the above-referenced methods, wherein Y is —C(R^{C})₂C(R^{C})₂R, —C(R^{C})═C(R^{C})R or —C≡CR.

In certain embodiments, the present invention relates to any one of the above-referenced methods, wherein Y is trans-vinylene.

In certain embodiments, the present invention relates to any one of the above-referenced methods, wherein R is

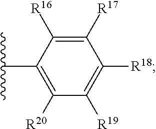

wherein R¹⁶, R¹⁷, R¹⁸, R¹⁹ and R²⁰ are independently selected from the group consisting of hydrogen, prenyl, alkenyl, alkoxy, alkyl, alkylcarbonyloxy, alkylthio, alkylcarbonylthio, alkynyl, amido, amidoalkyl, amino, aminoalkyl, carboxy, cyano, formyl, halo, haloalkoxy, haloalkyl, haloalkylthio, hydroxyl, hydroxyalkyl, mercapto, mercaptoalkyl, nitro, silyl, silyloxy and silyloxyalkyl; provided at least one R¹⁶, R¹⁷, R¹⁸, R¹⁹ and R²⁰ is prenyl.

In certain embodiments, the present invention relates to any one of the above-referenced methods, wherein R¹⁶ is hydrogen.

In certain embodiments, the present invention relates to any one of the above-referenced methods, wherein R¹⁷ is hydrogen, alkoxy, alkylcarbonyloxy, halo, haloalkoxy, haloalkyl, hydroxyl, hydroxyalkyl, silyloxy and silyloxyalkyl. In certain embodiments, the present invention relates to any one of the above-referenced methods, wherein R¹⁷ is alkoxy or hydroxyl. In certain embodiments, the present invention relates to any one of the above-referenced methods, wherein R¹⁷ is hydroxyl.

In certain embodiments, the present invention relates to any one of the above-referenced methods, wherein R¹⁸ is prenyl. In certain embodiments, the present invention relates to any one of the above-referenced methods, wherein R¹⁸

is

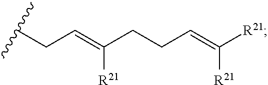

and R²¹ is hydrogen, alkyl or haloalkyl. In certain embodiments, the present invention relates to any one of the above-referenced methods, wherein R¹⁸ is geranyl.

In certain embodiments, the present invention relates to any one of the above-referenced methods, wherein R¹⁹ is hydrogen, alkoxy, alkylcarbonyloxy, halo, haloalkoxy, haloalkyl, hydroxyl, hydroxyalkyl, silyloxy and silyloxyalkyl. In certain embodiments, the present invention relates to any one of the above-referenced methods, wherein R¹⁹ is alkoxy or hydroxyl. In certain embodiments, the present invention relates to any one of the above-referenced methods, wherein R¹⁹ is hydroxyl.

In certain embodiments, the present invention relates to any one of the above-referenced methods, wherein R²⁰ is hydrogen.

In certain embodiments, the present invention relates to any one of the above-referenced methods, wherein Z is —C(R³)—.

In certain embodiments, the present invention relates to any one of the above-referenced methods, wherein R³ is hydrogen.

In certain embodiments, the present invention relates to any one of the above-referenced methods, wherein R⁴ is hydrogen, alkyl or haloalkyl. In certain embodiments, the present invention relates to any one of the above-referenced methods, wherein R⁴ is hydrogen.

In certain embodiments, the present invention relates to any one of the above-referenced methods, wherein R⁵ is hydrogen, alkyl or haloalkyl. In certain embodiments, the present invention relates to any one of the above-referenced methods, wherein R⁵ is hydrogen.

In certain embodiments, the present invention relates to any one of the above-referenced methods, wherein R⁶ is hydrogen, alkyl or haloalkyl. In certain embodiments, the present invention relates to any one of the above-referenced methods, wherein R⁶ is hydrogen.

In certain embodiments, the present invention relates to any one of the above-referenced methods, wherein R⁷ is hydrogen, alkyl or haloalkyl. In certain embodiments, the present invention relates to any one of the above-referenced methods, wherein R⁷ is hydrogen.

In certain embodiments, the present invention relates to any one of the above-referenced methods, wherein R⁸ is alkoxy, alkyl, alkylcarbonyloxy, halo, haloalkoxy, haloalkyl, hydrogen, hydroxyl, or silyloxy. In certain embodiments, the present invention relates to any one of the above-referenced methods, wherein R⁸ is hydrogen.

In certain embodiments, the present invention relates to any one of the above-referenced methods, wherein R⁹ is alkoxy, alkyl, alkylcarbonyloxy, halo, haloalkoxy, haloalkyl, hydrogen, hydroxyl, or silyloxy. In certain embodiments, the present invention relates to any one of the above-referenced methods, wherein R⁹ is hydrogen.

In certain embodiments, the present invention relates to any one of the above-referenced methods, wherein R¹⁰ alkoxy, alkyl, alkylcarbonyloxy, halo, haloalkoxy, haloalkyl, hydrogen, hydroxyl, or silyloxy. In certain embodiments, the present invention relates to any one of the above-referenced methods, wherein R¹⁰ is hydrogen.

In certain embodiments, the present invention relates to any one of the above-referenced methods, wherein R¹¹ is alkoxy, alkyl, alkylcarbonyloxy, halo, haloalkoxy, haloalkyl, hydroxyl, or silyloxy. In certain embodiments, the present invention relates to any one of the above-referenced methods, wherein R¹¹ is hydroxyl.

In certain embodiments, the present invention relates to any one of the above-referenced methods, wherein R¹² is alkoxy, alkyl, alkylcarbonyloxy, halo, haloalkoxy, haloalkyl, hydrogen, hydroxyl, or silyloxy. In certain embodiments, the present invention relates to any one of the above-referenced methods, wherein R¹² is hydrogen.

In certain embodiments, the present invention relates to any one of the above-referenced methods, wherein R¹³ alkoxy, alkyl, alkylcarbonyloxy, halo, haloalkoxy, haloalkyl, hydroxyl, or silyloxy. In certain embodiments, the present invention relates to any one of the above-referenced methods, wherein R¹³ is hydroxyl.

In certain embodiments, the present invention relates to any one of the above-referenced methods, wherein R¹⁴ is hydrogen, alkyl or haloalkyl. In certain embodiments, the present invention relates to any one of the above-referenced methods, wherein R¹⁴ is hydrogen.

In certain embodiments, the present invention relates to any one of the above-referenced methods, wherein R¹⁵ is hydrogen, alkyl or haloalkyl. In certain embodiments, the present invention relates to any one of the above-referenced methods, wherein R¹⁵ is hydrogen.

Certain aspects of the invention relate to a method of treating an oxysterol binding protein-related disease or condition, comprising the step of administering a therapeutically effective amount of a compound of formula V to a subject in need thereof; wherein the compound of formula V is represented by

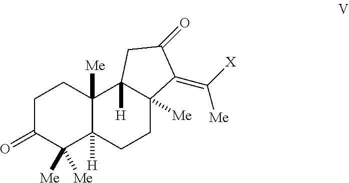

or a pharmaceutically acceptable salt, biologically active metabolite, solvate, hydrate, prodrug, enantiomer or stereoisomer thereof, wherein, independently for each occurrence,

X is

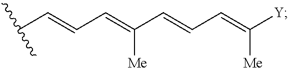

Y is —CH₂OR¹, —C(═O)R², —C(═O)OR³ or —C(═O)NHR³; and

R¹ is hydrogen, silyl, aralkyl, alkylcarboxy, arylcarboxy, heteroarylcarboxy, aralkylcarboxy or heteroaralkylcarboxy;

R² is hydrogen, alkyl, aryl, heteroaryl, aralkyl or heteroaralkyl; and

R³ is hydrogen, alkyl, aryl, heteroaryl, aralkyl, heteroaralkyl, alkylcarboxy, arylcarboxy, heteroarylcarboxy, aralkylcarboxy or heteroaralkylcarboxy.

In certain embodiments, the present invention relates to any one of the above-referenced methods, wherein Y is —CH₂OR¹. In certain embodiments, the present invention relates to any one of the above-referenced methods, wherein Y is —C(═O)R² In certain embodiments, the present invention relates to any one of the above-referenced methods, wherein Y is —C(═O)OR³. In certain embodiments, the present invention relates to any one of the above-referenced methods, wherein Y is —C(═O)NHR³.

In certain embodiments, the present invention relates to any one of the above-referenced methods, wherein R¹ is hydrogen. In certain embodiments, the present invention relates to any one of the above-referenced methods, wherein R¹ is —C(═O)CH₃. In certain embodiments, the present invention relates to any one of the above-referenced methods, wherein R¹ is silyl.

In certain embodiments, the present invention relates to any one of the above-referenced methods, wherein R² is alkyl. In certain embodiments, the present invention relates to any one of the above-referenced methods, wherein R² is aralkyl or heteroaralkyl.

In certain embodiments, the present invention relates to any one of the above-referenced methods, wherein R³ is hydrogen. In certain embodiments, the present invention relates to any one of the above-referenced methods, wherein R³ is alkyl, aryl, heteroaryl, aralkyl, heteroaralkyl, alkylcarboxy, arylcarboxy, heteroarylcarboxy, aralkylcarboxy or heteroaralkylcarboxy.

In certain embodiments, the present invention relates to any one of the above-referenced methods, wherein Y is —C(═O)OR³; and R³ is hydrogen.

In certain embodiments, the invention relates to any one of the above-referenced methods, wherein said oxysterol binding protein is OSBP1.

In certain embodiments, the invention relates to any one of the above-referenced methods, wherein the disease atherosclerosis.

In certain embodiments, the invention relates to the any of the above-referenced methods, wherein the disease Alzheimer's disease.

In certain embodiments, the invention relates to the any of the above-referenced methods, wherein the disease is cancer. Because CRAMs may be highly selective for tumors that have lost p21 expression, an event that often occurs at later stages in the clinical progression of some types of tumors [42], CRAMs might play a role as a new last line of defense in the treatment of cancer: as tumors become resistant to standard therapy through loss of p21, these tumors could be become more sensitive to the CRAMs. Therefore, in certain embodiments, the invention relates to any one of the above-referenced methods, wherein the disease is a p-21 deficient cancer.

Exemplary cancers that may be treated include cancers selected from the group consisting of Acute Lymphoblastic Leukemia; Acute Lymphoblastic Leukemia; Acute Myeloid Leukemia; Acute Myeloid Leukemia; Adrenocortical Carcinoma Adrenocortical Carcinoma; AIDS-Related Cancers; AIDS-Related Lymphoma; Anal Cancer; Astrocytoma, Childhood Cerebellar; Astrocytoma, Childhood Cerebral; Basal Cell Carcinoma, see Skin Cancer (non-Melanoma); Bile Duct Cancer, Extrahepatic; Bladder Cancer; Bladder Cancer; Bone Cancer, osteosarcoma/Malignant Fibrous Histiocytoma; Brain Stem Glioma; Brain Tumor; Brain Tumor, Brain Stem Glioma; Brain Tumor, Cerebellar Astrocytoma; Brain Tumor, Cerebral Astrocytoma/Malignant Glioma; Brain Tumor, Ependymoma; Brain Tumor, Medulloblastoma; Brain Tumor, Supratentorial Primitive Neuroectodermal Tumors; Brain Tumor, Visual Pathway and Hypothalamic Glioma; Brain Tumor; Breast Cancer; Breast Cancer and Pregnancy; Breast Cancer; Breast Cancer, Male; Bronchial Adenomas/Carcinoids; Burkitt's Lymphoma; Carcinoid Tumor; Carcinoid Tumor, Gastrointestinal; Carcinoma of Unknown Primary; Central Nervous System Lymphoma, Primary; Cerebellar Astrocytoma; Cerebral Astrocytoma/Malignant Glioma; Cervical Cancer; Childhood Cancers; Chronic Lymphocytic Leukemia; Chronic Myelogenous Leukemia; Chronic Myeloproliferative Disorders; Colon Cancer; Colorectal Cancer; Cutaneous T-Cell Lymphoma, see Mycosis Fungoides and Sezary Syndrome; Endometrial Cancer; Ependymoma; Esophageal Cancer; Esophageal Cancer; Ewing's Family of Tumors; Extracranial Germ Cell Tumor; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer; Gastrointestinal Carcinoid Tumor; Germ Cell Tumor, Extracranial; Germ Cell Tumor, Extragonadal; Germ Cell Tumor, Ovarian; Gestational Trophoblastic Tumor; Glioma; Glioma, Childhood Brain Stem; Glioma, Childhood Cerebral Astrocytoma; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma; Hodgkin's Lymphoma; Hodgkin's Lymphoma During Pregnancy; Hypopharyngeal Cancer; Hypothalamic and Visual Pathway Glioma; Intraocular Melanoma; Islet Cell Carcinoma (Endocrine Pancreas); Kaposi's Sarcoma; Kidney (Renal Cell) Cancer; Kidney Cancer; Laryngeal Cancer; Laryngeal Cancer; Leukemia, Acute Lymphoblastic; Leukemia, Acute Lymphoblastic; Leukemia, Acute Myeloid; Leukemia, Acute Myeloid; Leukemia, Chronic Lymphocytic; Leukemia; Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoma, AIDS-Related; Lymphoma, Burkitt's; Lymphoma, Cutaneous T-Cell, see Mycosis Fungoides and Sezary Syndrome; Lymphoma, Hodgkin's; Lymphoma, Hodgkin's; Lymphoma, Hodgkin's During Pregnancy; Lymphoma, Non-Hodgkin's; Lymphoma, Non-Hodgkin's; Lymphoma, Non-Hodgkin's During Pregnancy; Lymphoma, Primary Central Nervous System; Macroglobulinemia, Waldenstrom's; Malignant Fibrous Histiocytoma of Bone/Osteosarcoma; Medulloblastoma; Melanoma; Melanoma, Intraocular (Eye); Merkel Cell Carcinoma; Mesothelioma, Adult Malignant; Mesothelioma; Metastatic Squamous Neck Cancer with Occult Primary; Multiple Endocrine Neoplasia Syndrome; Multiple Myeloma/Plasma Cell Neoplasm' Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer; Neuroblastoma; Non-Hodgkin's Lymphoma; Non-Hodgkin's Lymphoma; Non-Hodgkin's Lymphoma During Pregnancy; Non-Small Cell Lung Cancer; Oral Cancer; Oral Cavity Cancer, Lip and; Oropharyngeal Cancer; Osteosarcoma/Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer; Pancreatic Cancer, Islet Cell; Paranasal Sinus and Nasal Cavity Cancer; Parathyroid Cancer; Penile Cancer; Pheochromocytoma; Pineoblastoma and Supratentorial Primitive Neuroectodermal Tumors; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Prostate Cancer; Rectal Cancer; Renal Cell (Kidney) Cancer; Renal Cell (Kidney) Cancer; Renal Pelvis and Ureter, Transitional Cell Cancer; Retinoblastoma; Rhabdomyosarcoma; Salivary Gland Cancer; Salivary Gland Cancer; Sarcoma, Ewing's Family of Tumors; Sarcoma, Kaposi's; Sarcoma, Soft Tissue; Sarcoma, Soft Tissue; Sarcoma, Uterine; Sezary Syndrome; Skin Cancer (non-Melanoma); Skin Cancer; Skin Cancer (Melanoma); Skin Carcinoma, Merkel Cell; Small Cell Lung Cancer; Small Intestine Cancer; Soft Tissue Sarcoma; Soft Tissue Sarcoma; Squamous Cell Carcinoma, see Skin Cancer (non-Melanoma); Squamous Neck Cancer with Occult Primary, Metastatic; Stomach (Gastric) Cancer; Stomach (Gastric) Cancer; Supratentorial Primitive Neuroectodermal Tumors; T-Cell Lymphoma, Cutaneous, see Mycosis Fungoides and Sezary Syndrome; Testicular Cancer; Thymoma; Thymoma and Thymic Carcinoma; Thyroid Cancer; Thyroid Cancer; Transitional Cell Cancer of the Renal Pelvis and Ureter; Trophoblastic Tumor, Gestational; Unknown Primary Site, Carcinoma of; Unknown Primary Site, Cancer of; Unusual Cancers of Childhood; Ureter and Renal Pelvis, Transitional Cell Cancer; Urethral Cancer; Uterine Cancer, Endometrial; Uterine Sarcoma; Vaginal Cancer; Visual Pathway and Hypothalamic Glioma; Vulvar Cancer; Waldenstrom's Macroglobulinemia; Wilms' Tumor; and Women's Cancers.

### DEFINITIONS

In this invention, the following definitions are applicable:

A “therapeutically effective amount” is an amount of a compound of the invention or a combination of two or more such compounds, which inhibits, totally or partially, the progression of the condition or alleviates, at least partially, one or more symptoms of the condition. A therapeutically effective amount can also be an amount which is prophylactically effective. The amount which is therapeutically effective will depend upon the patient's size and gender, the condition to be treated, the severity of the condition and the result sought. For a given patient, a therapeutically effective amount can be determined by methods known to those of skill in the art.

“Physiologically acceptable salts” refers to those salts which retain the biological effectiveness and properties of the free bases and which are obtained by reaction with inorganic acids, for example, hydrochloric acid, hydrobromic acid, sulfuric acid, nitric acid, and phosphoric acid or organic acids such as sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, tartaric acid (e.g., (+) or (−)-tartaric acid or mixtures thereof), amino acids (e.g., (+) or (−)-amino acids or mixtures thereof), and the like. These salts can be prepared by methods known to those skilled in the art.

Certain compounds of the invention which have acidic substituents may exist as salts with pharmaceutically acceptable bases. The present invention includes such salts. Examples of such salts include sodium salts, potassium salts, lysine salts and arginine salts. These salts may be prepared by methods known to those skilled in the art.

Certain compounds of the invention and their salts may exist in more than one crystal form and the present invention includes each crystal form and mixtures thereof.

Certain compounds of the invention and their salts may also exist in the form of solvates, for example hydrates, and the present invention includes each solvate and mixtures thereof.

Certain compounds of the invention may contain one or more chiral centers, and exist in different optically active forms. When compounds of the invention contain one chiral center, the compounds exist in two enantiomeric forms and the present invention includes both enantiomers and mixtures of enantiomers, such as racemic mixtures. The enantiomers may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step may be used to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a compound of the invention contains more than one chiral center, it may exist in diastereoisomeric forms. The diastereoisomeric compounds may be separated by methods known to those skilled in the art, for example chromatography or crystallization and the individual enantiomers may be separated as described above. The present invention includes each diastereoisomer of compounds of the invention and mixtures thereof.

Certain compounds of the invention may exist in different tautomeric forms or as different geometric isomers, and the present invention includes each tautomer and/or geometric isomer of compounds of the invention and mixtures thereof.

Certain compounds of the invention may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of the invention and mixtures thereof.

Certain compounds of the invention may exist in zwitterionic form and the present invention includes each zwitterionic form of compounds of the invention and mixtures thereof.

As used herein the term “pro-drug” refers to an agent which is converted into the parent drug in vivo by some physiological chemical process (e.g., a prodrug on being brought to the physiological pH is converted to the desired drug form). Pro-drugs are often useful because, in some situations, they may be easier to administer than the parent drug. They may, for instance, be bioavailable by oral administration whereas the parent drug is not. The prodrug may also have improved solubility in pharmacological compositions over the parent drug. An example, without limitation, of a pro-drug would be a compound of the present invention wherein it is administered as an ester (the “pro-drug”) to facilitate transmittal across a cell membrane where water solubility is not beneficial, but then it is metabolically hydrolyzed to the carboxylic acid once inside the cell where water solubility is beneficial. Pro-drugs have many useful properties. For example, a pro-drug may be more water soluble than the ultimate drug, thereby facilitating intravenous administration of the drug. A pro-drug may also have a higher level of oral bioavailability than the ultimate drug. After administration, the prodrug is enzymatically or chemically cleaved to deliver the ultimate drug in the blood or tissue.

Exemplary pro-drugs upon cleavage release the corresponding free acid, and such hydrolyzable ester-forming residues of the compounds of this invention include but are not limited to carboxylic acid substituents (e.g., —C(O)₂H or a moiety that contains a carboxylic acid) wherein the free hydrogen is replaced by (C₁-C₄)alkyl, (C₂-C₁₂)alkanoyloxymethyl, (C₄-C₉)1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as (3-dimethylaminoethyl), carbamoyl-(C₁-C₂)alkyl, N,N-di(C₁-C₂)-alkylcarbamoyl-(C₁-C₂)alkyl and piperidino-, pyrrolidino- or morpholino(C₂-C₃)alkyl.

Other exemplary pro-drugs release an alcohol of a compound of the invention wherein the free hydrogen of a hydroxyl substituent is replaced by (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyl-oxymethyl, N—(C₁-C₆)alkoxycarbonylamino-methyl, succinoyl, (C₁-C₆)alkanoyl, α-amino(C₁-C₄)alkanoyl, arylactyl and α-aminoacyl, or α-aminoacyl-α-aminoacyl wherein said α-aminoacyl moieties are independently any of the naturally occurring L-amino acids found in proteins, —P(O)(OH)₂, —P(O)(O(C₁-C₆)alkyl)₂ or glycosyl (the radical resulting from detachment of the hydroxyl of the hemiacetal of a carbohydrate).

For purposes of this invention, the chemical elements are identified in accordance with the Periodic Table of the Elements, CAS version, Handbook of Chemistry and Physics, 67th Ed., 1986-87, inside cover.

The articles “a” and “an” are used herein to refer to one or to more than one (i.e., to at least one) of the grammatical object of the article. By way of example, “an element” means one element or more than one element.

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy” means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxycarbonyl” means an alkoxy group, as defined herein, appended to the parent molecular moiety through a carbonyl group, represented by —C(═O)—, as defined herein. Representative examples of alkoxycarbonyl include, but are not limited to, methoxycarbonyl, ethoxycarbonyl, and tert-butoxycarbonyl.

The term “alkoxysulfonyl” as used herein, means an alkoxy group, as defined herein, appended to the parent molecular moiety through a sulfonyl group, as defined herein. Representative examples of alkoxysulfonyl include, but are not limited to, methoxysulfonyl, ethoxysulfonyl and propoxysulfonyl.

The term “arylalkoxy” and “heteroalkoxy” as used herein, means an aryl group or heteroaryl group, as defined herein, appended to the parent molecular moiety through an alkoxy group, as defined herein. Representative examples of arylalkoxy include, but are not limited to, 2-chlorophenylmethoxy, 3-trifluoromethylethoxy, and 2,3-methylmethoxy.

The term “arylalkyl” as used herein, means an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkyl” means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, and n-hexyl.

The term “alkylene,” is art-recognized, and as used herein, pertains to a bidentate moiety obtained by removing two hydrogen atoms, either both from the same carbon atom, or one from each of two different carbon atoms, of a hydrocarbon compound, which may be aliphatic or alicyclic, or a combination thereof, and which may be saturated, partially unsaturated, or fully unsaturated. Examples of linear saturated C₁₋₁₀alkylene groups include, but are not limited to, —(CH₂)ₙ— where n is an integer from 1 to 10, for example, —CH₂— (methylene), —CH₂CH₂— (ethylene), —CH₂CH₂CH₂— (propylene), —CH₂CH₂CH₂CH₂— (butylene), —CH₂CH₂CH₂CH₂CH₂— (pentylene) and —CH₂CH₂CH₂CH₂CH₂CH₂— (hexylene). Examples of branched saturated C₁₋₁₀alkylene groups include, but are not limited to, —CH(CH₃)—, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—. Examples of linear partially unsaturated C₁₋₁₀alkylene groups include, but are not limited to, —CH═CH— (vinylene), —CH═CH—CH₂—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, and —CH═CH—CH₂—CH₂—CH═CH—. Examples of branched partially unsaturated C₁₋₁₀alkylene groups include, but are not limited to, —C(CH₃)═CH—, —C(CH₃)═CHCH₂—, and —CH═CH—CH(CH₃)—. Examples of alicyclic saturated C₁₋₁₀alkylene groups include, but are not limited to, cyclopentylene (e.g., cyclopent-1,3-ylene), and cyclohexylene (e.g., cyclohex-1,4-ylene). Examples of alicyclic partially unsaturated C₁₋₁₀alkylene groups include, but are not limited to, cyclopentenylene (e.g., 4-cyclopenten-1,3-ylene), and cyclohexenylene (e.g., 2-cyclohexen-1,4-ylene, 3-cyclohexen-1,2-ylene, and 2,5-cyclohexadien-1,4-ylene).

The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonyloxy” and “arylcarbonyloxy” as used herein, means an alkylcarbonyl or arylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy. Representative examples of arylcarbonyloxy include, but are not limited to phenylcarbonyloxy.

The term “alkylsulfonyl” as used herein, means an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylthio” as used herein, means an alkyl group, as defined herein, appended to the parent molecular moiety through a sulfur atom. Representative examples of alkylthio include, but are not limited, methylthio, ethylthio, tert-butylthio, and hexylthio. The terms “arylthio,” “alkenylthio” and “arylakylthio,” for example, are likewise defined.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “amido” as used herein, means —NHC(═O)—, wherein the amido group is bound to the parent molecular moiety through the nitrogen. Examples of amido include alkylamido such as CH₃C(═O)N(H)— and CH₃CH₂C(═O)N(H)—.

The term “amino” as used herein, refers to radicals of both unsubstituted and substituted amines appended to the parent molecular moiety through a nitrogen atom. The two groups are each independently hydrogen, alkyl, alkylcarbonyl, alkylsulfonyl, arylcarbonyl, or formyl. Representative examples include, but are not limited to methylamino, acetylamino, and acetylmethylamino.

The term “aromatic” refers to a planar or polycyclic structure characterized by a cyclically conjugated molecular moiety containing 4n+2 electrons, wherein n is the absolute value of an integer. Aromatic molecules containing fused, or joined, rings also are referred to as bicylic aromatic rings. For example, bicyclic aromatic rings containing heteroatoms in a hydrocarbon ring structure are referred to as bicyclic heteroaryl rings.

The term “aryl,” as used herein, means a phenyl group or a naphthyl group. The aryl groups of the present invention can be optionally substituted with one, two, three, four, or five substituents independently selected from the group consisting of deuterium, tritium, alkenyl, alkoxy, alkoxycarbonyl, alkoxysulfonyl, alkyl, alkylcarbonyl, alkylcarbonyloxy, alkylsulfonyl, alkylthio, alkynyl, amido, amino, carboxy, cyano, formyl, halo, haloalkoxy, haloalkyl, hydroxyl, hydroxyalkyl, mercapto, nitro, silyl and silyloxy.

The term “arylalkyl” or “aralkyl” as used herein, means an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “arylalkoxy” or “arylalkyloxy” as used herein, means an arylalkyl group, as defined herein, appended to the parent molecular moiety through an oxygen. The term “heteroarylalkoxy” as used herein, means an heteroarylalkyl group, as defined herein, appended to the parent molecular moiety through an oxygen.

The term “arylcarbonyl” as used herein, means an aryl group, as defined herein, appended to the parent molecular moiety through a carbonyl group, as defined herein. Representative examples of arylcarbonyl include, but are not limited to, benzoyl and naphthoyl.

The term “aryloxy” as used herein, means an aryl group, as defined herein, appended to the parent molecular moiety through an oxygen. The term “heteroaryloxy” as used herein, means a heteroaryl group, as defined herein, appended to the parent molecular moiety through an oxygen.

The term “carbonyl” as used herein, means a —C(═O)— group.

The term “carboxy” as used herein, means a —CO₂H group.

The term “cycloalkyl” as used herein, means monocyclic or multicyclic (e.g., bicyclic, tricyclic, etc.) hydrocarbons containing from 3 to 12 carbon atoms that is completely saturated or has one or more unsaturated bonds but does not amount to an aromatic group. Examples of a cycloalkyl group include cyclopropyl, cyclobutyl, cyclopentyl, cyclopentenyl, cyclohexyl and cyclohexenyl.

The term “cycloalkoxy” as used herein, means a cycloalkyl group, as defined herein, appended to the parent molecular moiety through an oxygen.

The term “cyano” as used herein, means a —CN group.

The term “formyl” as used herein, means a —C(═O)H group.

The term “halo” or “halogen” means —Cl, —Br, —I or —F.

The term “haloalkoxy” as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl” means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heterocyclyl”, as used herein, include non-aromatic, ring systems, including, but not limited to, monocyclic, bicyclic and tricyclic rings, which can be completely saturated or which can contain one or more units of unsaturation, for the avoidance of doubt, the degree of unsaturation does not result in an aromatic ring system) and have 3 to 12 atoms including at least one heteroatom, such as nitrogen, oxygen, or sulfur. For purposes of exemplification, which should not be construed as limiting the scope of this invention, the following are examples of heterocyclic rings: azepines, azetidinyl, morpholinyl, oxopiperidinyl, oxopyrrolidinyl, piperazinyl, piperidinyl, pyrrolidinyl, quinicludinyl, thiomorpholinyl, tetrahydropyranyl and tetrahydrofuranyl. The heterocyclyl groups of the invention are substituted with 0, 1, 2, or 3 substituents independently selected from deuterium, tritium, alkenyl, alkoxy, alkoxycarbonyl, alkoxysulfonyl, alkyl, alkylcarbonyl, alkylcarbonyloxy, alkylsulfonyl, alkylthio, alkynyl, amido, amino, carboxy, cyano, formyl, halo, haloalkoxy, haloalkyl, hydroxyl, hydroxyalkyl, mercapto, nitro, silyl and silyloxy.

The term “heteroaryl” as used herein, include aromatic ring systems, including, but not limited to, monocyclic, bicyclic and tricyclic rings, and have 3 to 12 atoms including at least one heteroatom, such as nitrogen, oxygen, or sulfur. For purposes of exemplification, which should not be construed as limiting the scope of this invention: azaindolyl, benzo(b)thienyl, benzimidazolyl, benzofuranyl, benzoxazolyl, benzothiazolyl, benzothiadiazolyl, benzotriazolyl, benzoxadiazolyl, furanyl, imidazolyl, imidazopyridinyl, indolyl, indolinyl, indazolyl, isoindolinyl, isoxazolyl, isothiazolyl, isoquinolinyl, oxadiazolyl, oxazolyl, purinyl, pyranyl, pyrazinyl, pyrazolyl, pyridinyl, pyrimidinyl, pyrrolyl, pyrrolo[2,3-d]pyrimidinyl, pyrazolo[3,4-d]pyrimidinyl, quinolinyl, quinazolinyl, triazolyl, thiazolyl, thiophenyl, tetrahydroindolyl, tetrazolyl, thiadiazolyl, thienyl, thiomorpholinyl, triazolyl or tropanyl. The heteroaryl groups of the invention are substituted with 0, 1, 2, or 3 substituents independently selected from deuterium, tritium, alkenyl, alkoxy, alkoxycarbonyl, alkoxysulfonyl, alkyl, alkylcarbonyl, alkylcarbonyloxy, alkylsulfonyl, alkylthio, alkynyl, amido, amino, carboxy, cyano, formyl, halo, haloalkoxy, haloalkyl, hydroxyl, hydroxyalkyl, mercapto, nitro, silyl and silyloxy.

The term “heteroarylalkyl” or “heteroaralkyl” as used herein, means a heteroaryl, as defined herein, appended to the parent molecular moiety through an alkyl group, as defined herein. Representative examples of heteroarylalkyl include, but are not limited to, pyridin-3-ylmethyl and 2-(thien-2-yl)ethyl.

The term “hydroxy” as used herein, means an —OH group.

The term “hydroxyalkyl” as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “mercapto” as used herein, means a —SH group.

The term “nitro” as used herein, means a —NO₂ group.

The term “prenyl” as used herein dimethylallyl, geranyl, neryl, farnesyl and geranylfarnesyl, as well as partially saturated equivalents thereof.

The term “prenylaryl” or “prenylheteroaryl” means a prenyl, as defined herein, appended to the parent molecular moiety through an aryl or heteroaryl, as defined herein. Representative examples of haloalkoxy include geranylphenyl and geranylpyridinyl.

The term “silyl” as used herein includes hydrocarbyl derivatives of the silyl (H₃Si—) group (i.e., (hydrocarbyl)₃Si—), wherein a hydrocarbyl groups are univalent groups formed by removing a hydrogen atom from a hydrocarbon, e.g., ethyl, phenyl. The hydrocarbyl groups can be combinations of differing groups which can be varied in order to provide a number of silyl groups, such as trimethylsilyl (TMS), tert-butyldiphenylsilyl (TBDPS), tert-butyldimethylsilyl (TBS/TBDMS), triisopropylsilyl (TIPS), and [2-(trimethylsilyl)ethoxy]methyl (SEM).

The term “silyloxy” as used herein means a silyl group, as defined herein, is appended to the parent molecule through an oxygen atom.

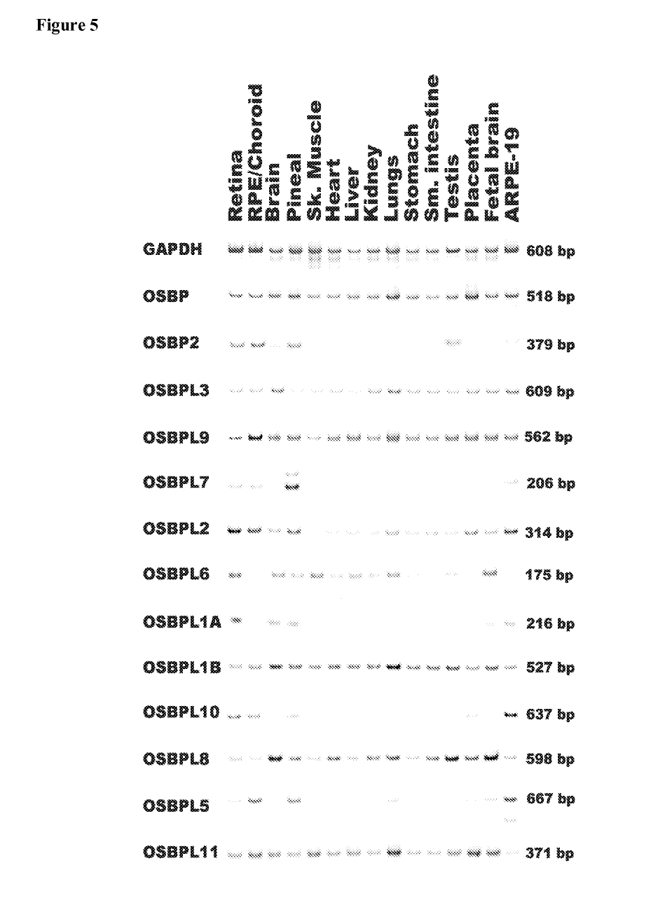

The term “oxysterol binding protein” as used herein includes oxysterol binding protein (OSBP or OSBP1) as well as oxysterol binding protein-related proteins (such as ORP2-11; for example, see FIG. 5). As used herein, a “carbohydrate” (or, equivalently, a “sugar”) is a saccharide (including monosaccharides, oligosaccharides and polysaccharides) and/or a molecule (including oligomers or polymers) derived from one or more monosaccharides, e.g., by reduction of carbonyl groups, by oxidation of one or more terminal groups to carboxylic acids, by replacement of one or more hydroxy group(s) by a hydrogen atom, an amino group, a thiol group or similar heteroatomic groups, etc. The term “carbohydrate” also includes derivatives of these compounds. Non-limiting examples of carbohydrates include allose (“All”), altrose (“Alt”), arabinose (“Ara”), erythrose, erythrulose, fructose (“Fm”), fucosamine (“FucN”), fucose (“Fuc”), galactosamine (“GalN”), galactose (“Gal”), glucosamine (“GlcN”), glucosaminitol (“GlcN-ol”), glucose (“Glc”), glyceraldehyde, 2,3-dihydroxypropanal, glycerol (“Gro”), propane-1,2,3-triol, glycerone (“1,3-dihydroxyacetone”), 1,3-dihydroxypropanone, gulose (“Gul”), idose (“Ido”), lyxose (“Lyx”), mannosamine (“ManN”), mannose (“Man”), psicose (“Psi”), quinovose (“Qui”), quinovosamine, rhamnitol (“Rha-ol”), rhamnosamine (“RhaN”), rhamnose (“Rha”), ribose (“Rib”), ribulose (“Rul”), sialic acid (“Sia” or “Neu”), sorbose (“Sor”), tagatose (“Tag”), talose (“Tal”), tartaric acid, erythraric/threaric acid, threose, xylose (“Xyl”), xylulose (“Xul”), and combinations thereof. In some cases, the carbohydrate may be a pentose (i.e., having 5 carbons) or a hexose (i.e., having 6 carbons); and in certain instances, the carbohydrate may be an oligosaccharide comprising pentose and/or hexose units, e.g., including those described above. The term carbohydate also includes carbohydrates wherein the pendant hydroxyl or amino groups are protected. For example, a hydroxy group may be protected as an ether (—OR) or an ester (—OC(═O)R), wherein R is alkyl, aryl, heteroaryl, aralkyl or heteroaralkyl, for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc); and an amine group may be protected, for example, as an amide (—NRC(═O)R) or a urethane (—NRC(═O)OR), wherein R is alkyl, aryl, heteroaryl, aralkyl or heteroaralkyl, for example, as: a methyl amide (—NHC(═O)CH₃); a benzyloxy amide (—NHC(═O)OCH₂C₆H₅NHCbz); as a t-butoxy amide (—NHC═(═O)OC(CH₃)₃, —NHBoc); a 2-biphenyl-2-propoxy amide (—NHC(═O)OC(CH₃)₂C₆H₄C₆H₅NHBoc), as a 9-fluorenylmethoxy amide (—NHFmoc), as a 6-nitroveratryloxy amide (—NHNvoc), as a 2-trimethylsilylethyloxy amide (—NHTeoc), as a 2,2,2-trichloroethyloxy amide (—NHTroc), as an allyloxy amide (—NHAlloc), as a 2-(phenylsulfonyl)ethyloxy amide (—NHPsec); or, in suitable cases (e.g., cyclic amines), as a nitroxide radical.

A “monosaccharide,” is a carbohydrate or carbohydrate derivative that includes one saccharide unit. Similarly, a “disaccharide,” a “trisaccharide,” a “tetrasaccharide,” a “pentasaccharide,” etc. respectively has 2, 3, 4, 5, etc. saccharide units.

As used herein, the phrase “subject in need thereof” means a subject identified as in need of a therapy or treatment of the invention.

As used herein, the phrase “therapeutic effect” refers to a local or systemic effect in animals, particularly mammals, and more particularly humans, caused by an agent. The phrases “therapeutically-effective amount” and “effective amount” mean the amount of an agent that produces some desired effect in at least a sub-population of cells. A therapeutically effective amount includes an amount of an agent that produces some desired local or systemic effect at a reasonable benefit/risk ratio applicable to any treatment. For example, certain agents used in the methods of the present invention may be administered in a sufficient amount to produce a reasonable benefit/risk ratio applicable to such treatment.

As used herein, the term “treating” a disease in a subject or “treating” a subject having or suspected of having a disease refers to subjecting the subject to a pharmaceutical treatment, e.g., the administration of an agent, such that at least one symptom of the disease is decreased or prevented from worsening.

As used herein, an “oxysterol binding protein-related disease” is a disease which may be treated by administering a compound which modulates the activity of an oxysterol binding protein.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Evaluation of Molecules for Dependence on p21 Status

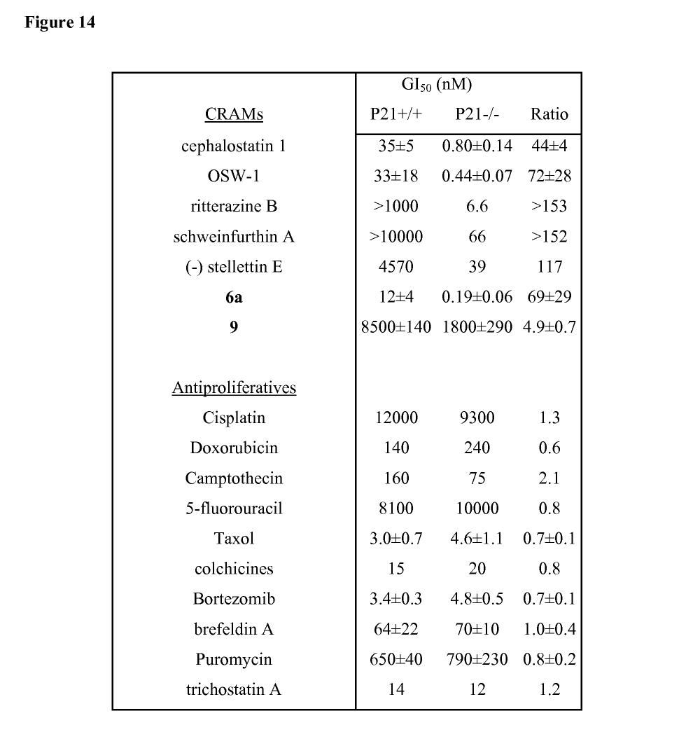

As noted above, it has been reported that the CRAM stellettin E (5) is 117 fold more active in HCT-116 colon cancer cell lines lacking the p21 protein. [5] Many of the other CRAMs have been tested and it has been determined that they also are highly selective for inhibiting the proliferation of HCT-116 p21^{-/-} cells versus HCT-116 p21^{+/+} cells (FIG. 14). This shared p21 differential cytotoxicity supports the idea of a common CRAM mechanism of action and likely similar target. For example, cephalostatin 1 has a differential GI₅₀ of 44 fold, OSW-1 has a differential GI₅₀ of 72 fold. In contrast many known antiproliferative/cytotoxic small molecules evaluated show little if any dependence on p21 status, as does an inactive derivative of OSW-1 (9) (FIG. 14). It appears that CRAMs may be among the most selective cytotoxic molecules yet identified in cells lacking p21. However, caution has to be exercised in concluding that compounds found to be selective for p21 deficient HCT-116 cells are truly synthetic lethal with p21. It was recently reported that compounds shown to be hypersensitive to HCT-116 p21^{-/-} cells was actually due to an overexpression of p53 in that cell line. [41] Subsequent siRNA knockdown of p53 eliminated the apparent p21 synthetic lethality. However, it has been determined that siRNA knockdown of p53 in HCT-116 p21^{-/-} cells does not alter the GI₅₀ of OSW-1 (data not shown), supporting CRAMs as being truly p21 synthetic lethal.

### Example 2

### Preparation of OSW-1 Derivatives and Affinity Chromatography

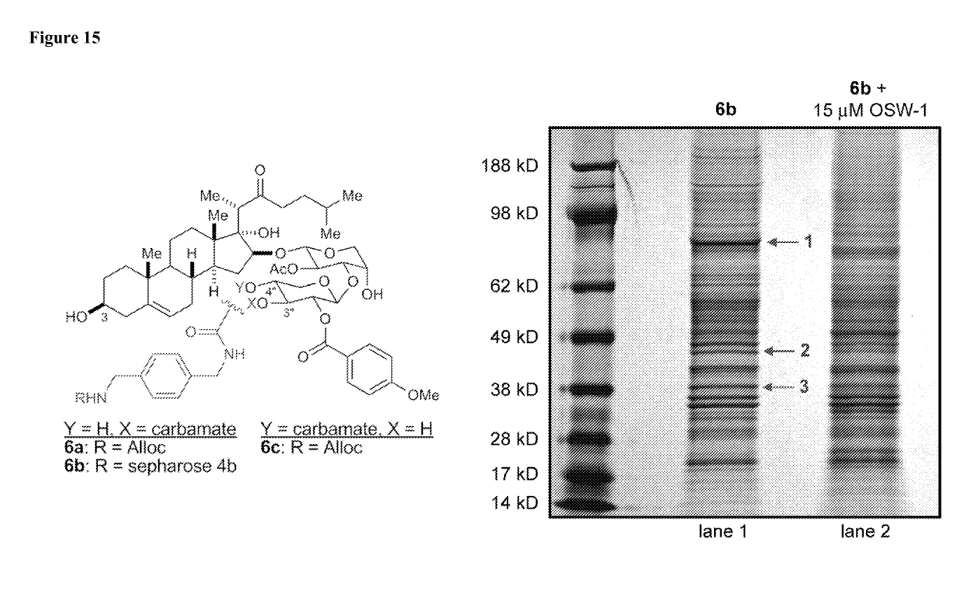

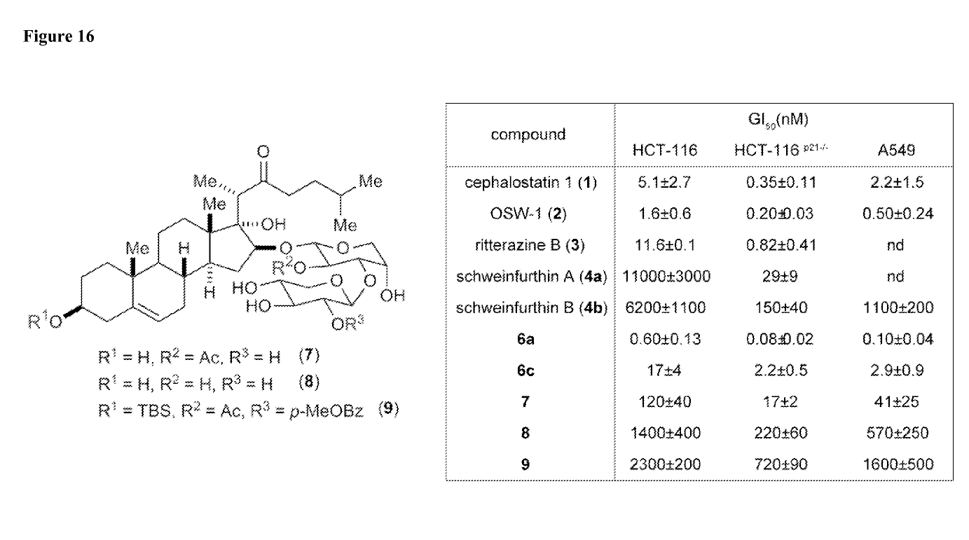

Starting with OSW-1, derivatives of OSW-1 that maintained their antiproliferative activity were prepared. Through rounds of non-selective carbamate forming reactions, separation, and cell viability testing, it was discovered that carbamate analog 6a (FIG. 15) retained all the activity of OSW-1 (GI₅₀=0.29 nM for 6a versus 0.23 nM of OSW-1 in HeLa cells) and it was even more active in other cell lines (FIG. 16). 6a also retained the p21^{-/-} differential cytotoxicity (FIG. 14). Along with the less active carbamate derivative 6c, OSW-1 analogs 7-9 were also prepared, thus affording a series of OSW-1 derived structures with activities covering nearly 4 orders of magnitude when evaluated in three different cancer cell lines (FIG. 16).

Using active OSW-1 analog 6a, described above, sepharose-linked affinity reagent 6b was prepared, and affinity chromatography experiments using HeLa cell S-100 lysates were performed. The lysates were incubated with the affinity matrix in the absence (lane 1) or presence (lane 2) of soluble (15 μM) OSW-1. Upon repeating the affinity chromatography experiment, only band 1, not bands 2 or 3, was enriched. Excision of band 1, trypsin digestion, and LC-MS/MS revealed this band as being oxysterol-binding protein (OSBP). [43] Subsequently lanes 1 and 2 were processed in their entirety to identify proteins competed in lane 2 that were not visible on the gel. This experiment revealed that ORP4 was also competed in lane 2, although the abundance was much lower than that of OSBP. The pulldown of OSBP by OSW-1 affinity reagent 6b, successfully repeated twice, strongly supports the conclusion that 6b binds OSBP, and more importantly, so does OSW-1, as revealed by the competition experiments. No other CRAMs were tested in affinity chromatography competition experiments, but described below are direct binding studies between CRAMs and OSBP. Since OSBP is the most abundant of the OSBP/ORPs in cells, it was the most abundant OSBP/ORP from the pulldown, and because its biology is the best characterized, further experiments based upon OSBP were performed, as detailed below.

### Example 3

### shRNA Knockdown of OSBP Assay

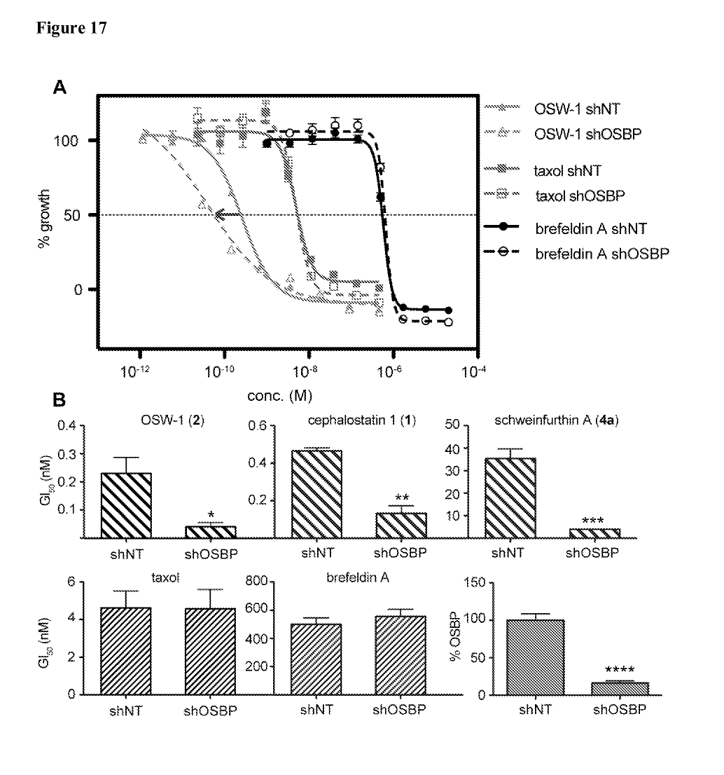

To determine whether the cytotoxicity of CRAMs depends on the amount of OSBP in cells, the effects of shRNA knockdown of OSBP on the growth inhibitory activity of CRAMs was measured. Treatment of HCT-116 p21^{-/-} cells with shRNA targeted to OSBP [24] caused an 84% reduction in the level of OSBP as measured by western blot (FIG. 17B, bottom right). Interestingly, knockdown of OSBP did not cause cell death nor was it growth inhibitory to this cell line. The GI₅₀ of OSW-1 in HCT-116 p21^{-/-} shNT cells (cells expressing non-targeting shRNA) is 0.23 nM. However, upon shRNA knockdown of OSBP by 84%, the GI₅₀ shifted by 5.6 fold, to 0.04 nM, demonstrating that OSBP knockdown sensitizes these cells to OSW-1 (see arrow in FIG. 17A).

It was then determined that OSBP knockdown does not sensitize cells to taxol, brefeldin A, or bortezomib (bortezomib data not shown) demonstrating that OSBP knockdown does not non-specifically render cells more sensitive to cytotoxic small molecules.

It was also observed that OSBP knockdown sensitized HCT-116 p21^{-/-} cells to three other CRAMs (ritterazine B data not shown) (FIG. 17B). Specifically, knockdown of OSBP sensitized HCT-116 p21^{-/-} cells to schweinfurthin A by 8.9 fold and to cephalostatin 1 by 3.5 fold as measured at the GI₅₀. In a second cell line, HeLa, the same sensitization of cells to OSW-1 by shRNA knockdown of OSBP was observed (data not shown), suggesting that the effects observed are not specific to HCT-116 p21^{-/-} cells.

Since shRNA knockdown of OSBP is not cytotoxic and because reducing the amount of OSBP sensitizes cells to CRAMs, it may mean that OSBP binds CRAMs in cells, but OSBP alone is not responsible for the cytotoxic activity of CRAMs. The change in sensitivity to CRAMs could be a consequence of eliminating a major CRAM-binding protein, but not one that alone mediates the cytotoxicity of CRAMs. It is possible that the target mediating cytotoxicity may be among the ORPs, or even a separate unidentified protein. However, data presented below strongly implicate OSBP/ORPs as the targets mediating cytotoxicity of CRAMs.

### Example 4

### Competitive Binding Assay

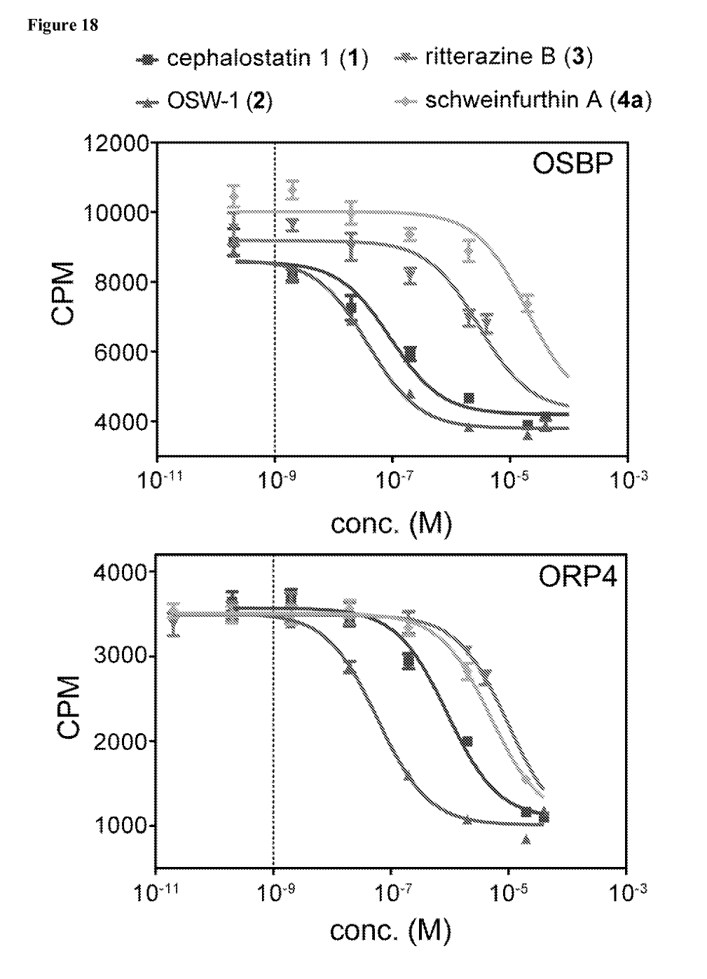

It has been determined that CRAMs inhibit the binding of radiolabelled 25-OHC ([³H]-25-OHC) to recombinant OSBP-myc-his and ORP4-myc-his overexpressed in HEK-293T lysate (FIG. 18). It is known that 25-hydroxycholesterol is a high affinity ligand for OSBP and ORP4. Increasing concentrations of CRAMs or OSW-1 analogs were incubated with 20 nM [³H]-25-OHC in OSBP-myc-his or ORP4-myc-his S100 lysate for 16 h at 4° C. after which unbound sterol was removed using charcoal-dextran and the bound sterol quantified by LCS (liquid scintillation counting).

It was found that OSW-1 and cephalostatin 1 are nearly as efficient as 25-OHC at displacing OSBP-bound [³H]-25-OHC, with Kᵢ-values of 21 nM and 50 nM respectively. At low micromolar-concentrations both ritterazine B and schweinfurthin A also displaced the bound ligand. CRAMs also displaced the radioligand from ORP4 and again OSW-1 (Kᵢ=32 nM) was found to be approximately equipotent to 25-OHC. These experiments provide evidence that CRAMs are ligands of OSBP and ORP4, and in the case of 1 and 2, they are high affinity ligands.

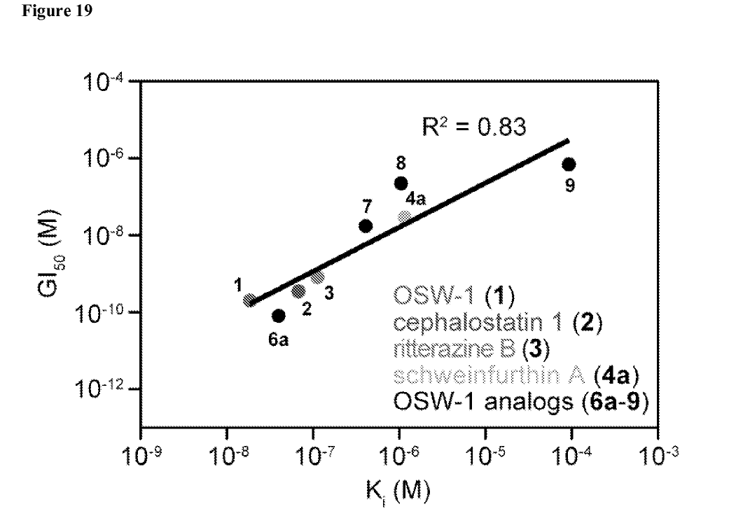

The Kᵢ values for the OSW-1 analogs (6a, 7-9) in competing for the binding of [³H]-25-OHC to OSBP have also been measured. These values were used in combination with those of the CRAMs to show that there is a high correlation between their Kᵢ-values and their cytotoxic activity (FIG. 19).

The Kᵢ/GI₅₀ correlation (FIG. 19) strongly suggests that the target(s) mediating cytotoxicity of CRAMs have sterol-binding domains highly similar to that of OSBP, which must be members of the OSBP/ORPs family. The sterol-binding domain of OSBP/ORPs is unique among proteins that bind sterols including LXR, Insig, NPC-1, and StARt proteins, meaning that only OSBP/ORPs would be expected to have a correlation between Kᵢ/GI₅₀.

### Example 5

### Sphingomyelin Synthesis Inhibition Assay

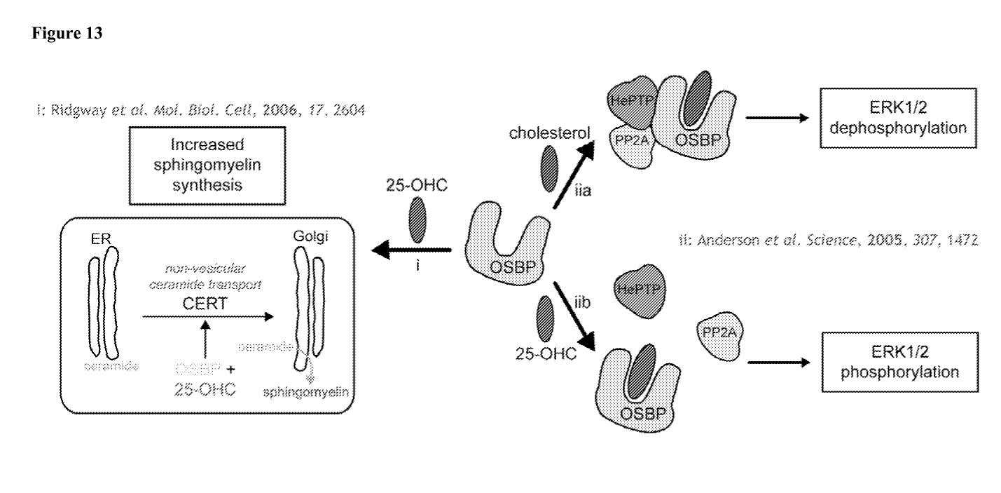

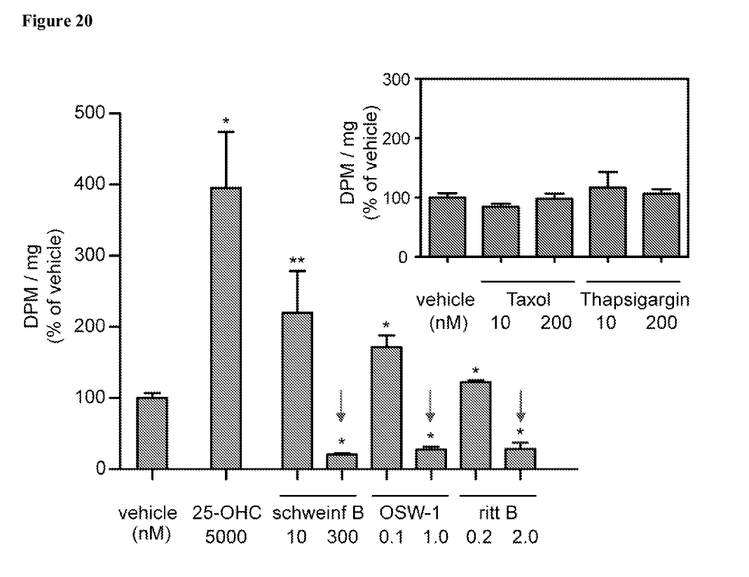

To begin to probe why CRAMs are cytotoxic and to determine if they perturb known OSBP activities, their effects on several published activities of OSBP have been tested. As mentioned above, addition of 25-OHC to cells causes an increase in the rate of sphingomyelin synthesis in an OSBP-dependent manner (FIG. 13, i). [22] Following a literature procedure [22], [³H]-serine pulselabelling experiments were performed (FIG. 20). Briefly, CHO-K1 cells were treated with CRAMs at their GI₁₀ and GI₉₀ concentrations for a total of 6 h including a 2 h [³H]-serine pulse. Then, isolation of sphingomyelin was performed, followed by scintillation counting to quantify the amount of tritium incorporated. At low doses, CRAMs modestly but reproducibly caused an increase in sphingomyelin biosynthesis, like 25-OHC. However, a dramatic lowering of sphingomyelin synthesis was induced at the CRAM GI₉₀-levels of dosing (see arrows, FIG. 20). Since OSBP is believed to help coordinate transport of ceramide from the ER to the Golgi for sphingomyelin biosynthesis [24], one possibility is that CRAMs are uniquely perturbing OSBP (or ORPs), blocking transport of ceramide, and causing a drop in sphingomyelin production. This could lead to a rise in ceramide levels in cells [44], which has been shown in several studies to cause apoptosis [45]. It follows that it is possible that the cytotoxic activity of CRAMs is due to a build up of toxic levels of ceramide in cells, via perturbation of OSBP/ORP function.

### Example 6

### Phosphorylation of ERK1/2 Assay

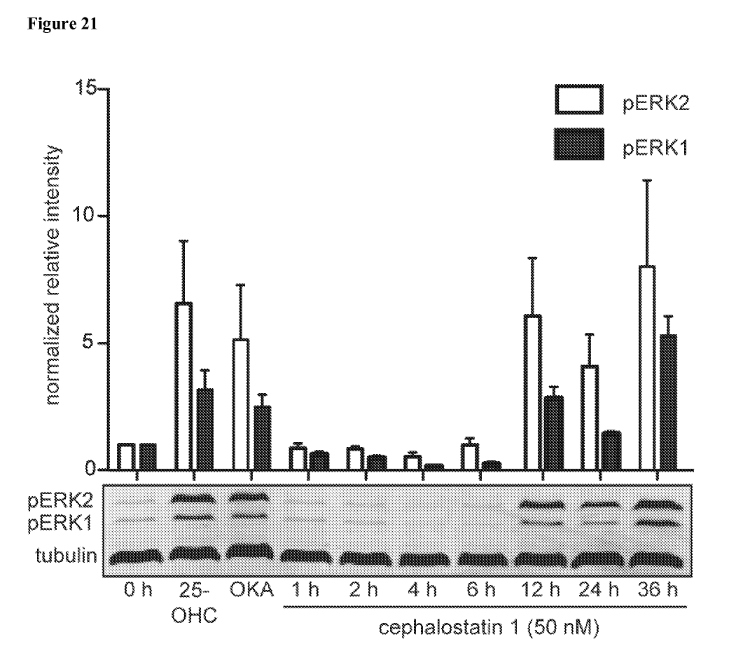

As another test of whether CRAMs perturb a known OSBP function, it was determined whether CRAMs induce changes to the phosphorylation state of ERK1/2. As mentioned above, 25-OHC binds OSBP and causes disruption of a complex between OSBP and the phosphatases HePTP and PP2A. [27] This blocks dephosphorylation of pERK1/2, leading to a persistence of pERK1/2. Opposing this effect is cholesterol, which binds OSBP, inducing formation of the OSBP-HePTP-PP2A ternary complex, causing ERK1/2 dephosphorylation (FIG. 13). [27] It has been discovered that in HCT-116 cells cephalostatin 1 caused a significant increase in pERK1/2 starting at 12 h and persisting for at least 24 h (FIG. 21). The increase in pERK1/2 levels induced by cephalostatin 1 was comparable to that caused by 25-OHC and okadaic acid (OKA), a general phosphatase inhibitor. OSW-1 (2) also induced an increase in pERK1/2 comparable to cephalostatin 1 (1) (data not shown). Since 25-OHC, which is not cytotoxic at doses up to 1000 times its OSBP K_{d}, had the same effect as CRAMs, it is unlikely this activity is responsible for the cytotoxicity of CRAMs.

### Example 7

### Immunofluorescence (IF) Experiments

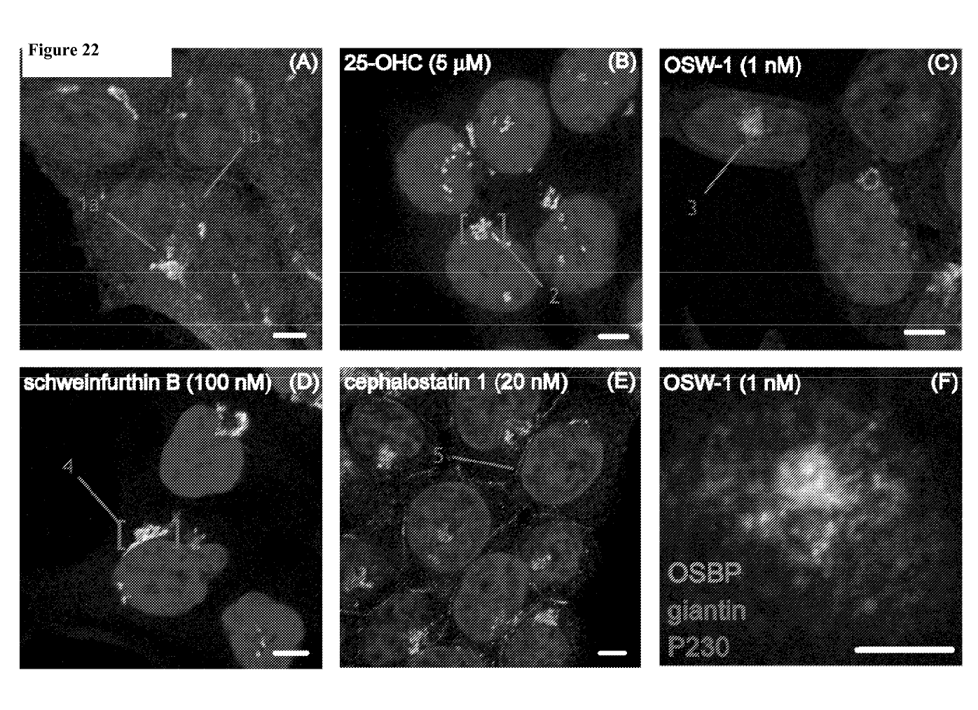

As another way to investigate how CRAMs may affect OSBP, immunofluorescence (IF) experiments were performed in HCT-116 p21^{+/+} cells to determine whether OSBP localization was altered upon treatment with different CRAMs. In untreated cells, OSBP resides largely at ER/cytoplasmic sites with a partial localization at the Golgi (FIG. 22A).

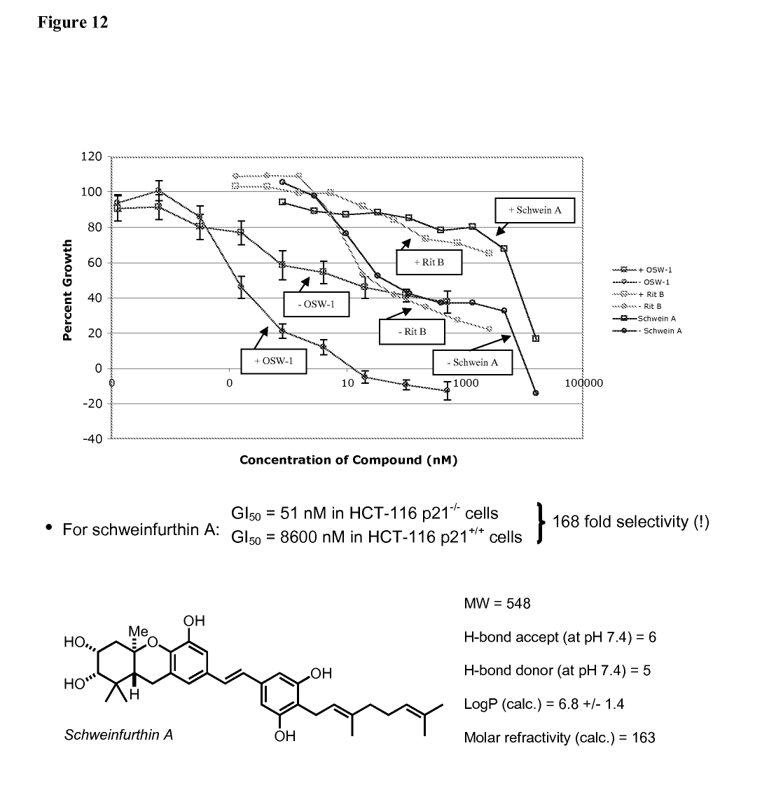

However, upon treatment with 25-OHC, OSBP translocates to the Golgi as displayed by colocalization with the Golgi marker p230 (see FIG. 22B). This translocation has been reported many times in the literature as an effect of 25-OHC on OSBP. [46] Treatment of cells with OSW-1 at 1 nM for 4 h caused a dramatic and unprecedented effect on OSBP localization (FIG. 22C). OSW-1 induced OSBP to shift from the cytoplasm to a tight cis/medial Golgi site as apparent by colocalization of OSBP with giantin. In this case p230 has been dispersed (see FIG. 122F). The translocation of OSBP induced by 2 is distinct from that of 25-OHC. Schweinfurthin B also induced OSBP translocation, but in a manner similar to the 25-OHC effect (FIG. 12D). Cephalostatin 1 induced OSBP to shift, but this time it caused a significant amount of OSBP to co-localize with the plasma membrane, again an effect not seen before with 25-OHC. These experiments tell us that CRAMs perturb OSBP localization in cells and that different CRAMs cause different effects, suggesting the possibility that CRAMs all perturb OSBP but in different ways. They also reveal that the effects on OSBP translocation by 1 and 2 are distinct from 25-OHC.

### Example 8

### Enantioselective Synthesis of Cephalostatin 1

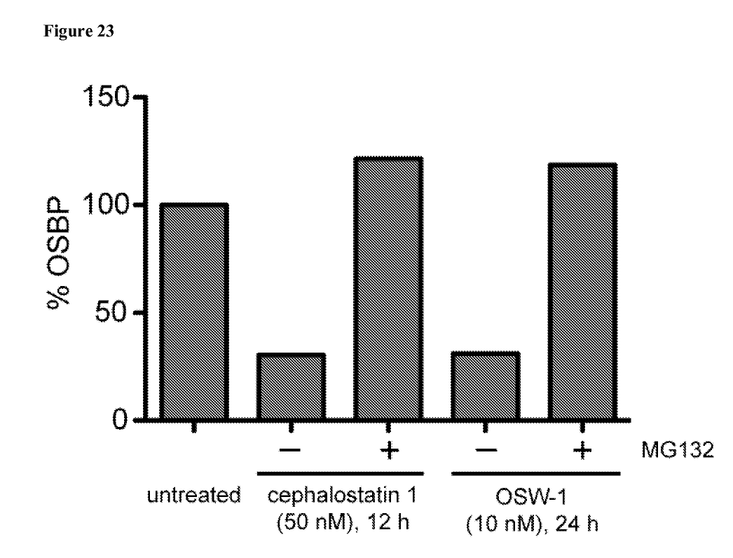

One unanticipated discovery from the IF studies was that, after several hours, CRAM treatment of cells caused a significant reduction in the intensity of the OSBP fluorescent signal. This could mean that CRAMs are inducing a reduction in OSBP protein levels: an effect not observed with 25-OHC. The amount of OSBP protein in cells exposed to CRAMs was measured by western blot. Indeed, it was discovered that cells treated with 1 or 2 induced a time-dependent reduction in OSBP protein levels (FIG. 23). Cephalostatin 1 induced a 70% reduction in OSBP at 12 h, and OSW-1 caused a 69% reduction in OSBP at 24 h. Treatment of CRAMs along with MG132, a proteasome inhibitor, resulted in complete rescue of OSBP levels (FIG. 13). This supports a situation where reduction of OSBP levels by 1 and 2 is a result of a proteasome-dependent degradation process. One possible scenario is that 1 and 2 are also causing protein level reductions of ORPs and this is the source of cytotoxicity of CRAMs.

### Example 9

### Synthesis of Cephalostatin 1 and OSW-1 Analogs

An enantioselective synthesis of cephalostatin 1 has been achieved. [47; hereby incorporated by reference in its entirety, including the supporting information] Key steps of this synthesis are a unique methyl group selective allylic oxidation, directed C—H hydroxylation of a sterol at C12, Au(I)-catalyzed 5-endo-dig cyclization, and a kinetic spiroketalization. This synthesis affords us access to 1 and analogs of 1 for the biological studies proposed in this grant.

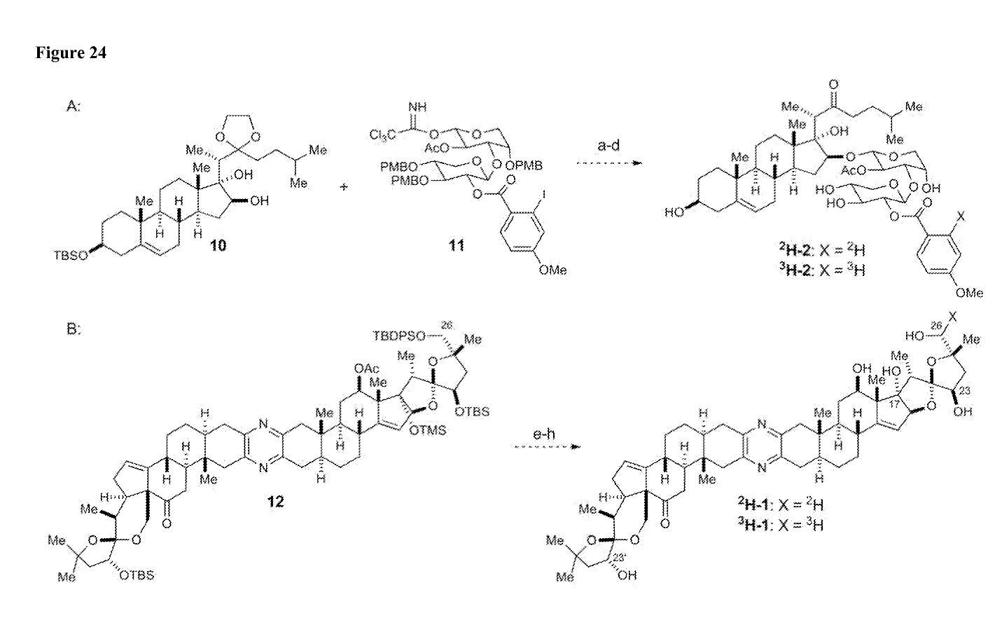

Synthesis of ³H-1 and ³H-2 will allow the measurement of their dissociation constants (K_{d}) to the OSBP/ORPs. Synthesis of ³H-1 and ³H-2 is also important since some of the ORPs may not bind 25-OHC preventing competitive displacement experiments. ³H—OSW-1 (FIG. 24, ³H-2) could be prepared by constructing the iodinated p-methoxybenzoate disaccharide fragment 11. To access 11, could employ the known route [48] substituting 2-iodo-4-methoxybenzoic acid in the acylation of the xylose C2 hydroxyl. This chemistry has already been used to prepare larger quantities of 2. Glycosylation and deprotection, as reported [48], will deliver iodinated OSW-1 analog. Isotope incorporation will be accomplished through reduction with Pd/C and D₂. It has been observed that hydrogenation of the C₅-C₆ olefin is very sluggish, suggesting that it should be possuble to selectively reduce the aryl iodide.

One could also prepare ³H-cephalostatin 1 (FIG. 24, ³H-1) by selective deprotection of the C26 OTBDPS group of 12, a late stage intermediate of the total synthesis referenced above. Then, oxidation of the primary alcohol to the aldehyde, reduction with NaBD₄, and finally global deprotection with TBAF would afford ³H-1.

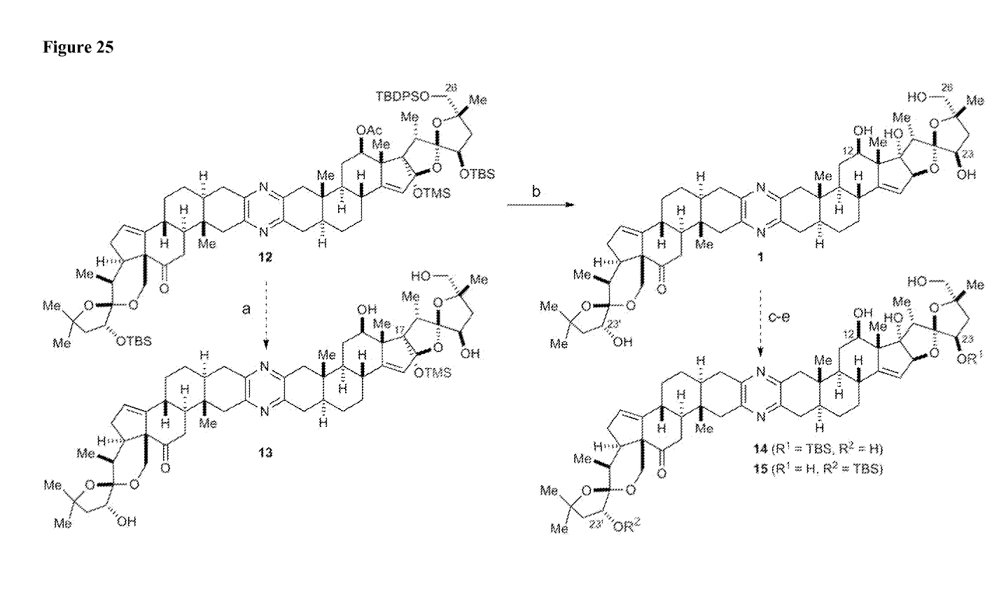

Using the above-notes synthesis route to 1, one could prepare differentially protected analogs of 1 that may exhibit different GI₅₀s (e.g., those in FIG. 25). Starting from protected cephalostatin 1 (12) one might perform a TBAF-deprotection at RT to afford TMS-protected tertiary alcohol 13. It has been observed that the C17 tertiary carbinol was last to deprotect. From synthetic cephalostatin 1 (1) one might selectively protect the primary hydroxyl group at C26 as an acetate. [50] TBS-protection under standard conditions might then deliver a mixture of compounds having secondary TBS-ethers at positions C23 and C23′. It has been observed that the C12 secondary carbinol is resistant to silylation due to steric hindrance. Therefore, deprotection of the acetate group should result in mono-TBS protected cephalostatin 1 analogs 14 and 15. These analogs could be subjected to a standard cell viability assays in HCT-116, HCT-116 HeLa, and HEK-293T cells.

### Example 10

### Development of a OSBP/ORP Binding Scintillation Proximity Assay (SPA)

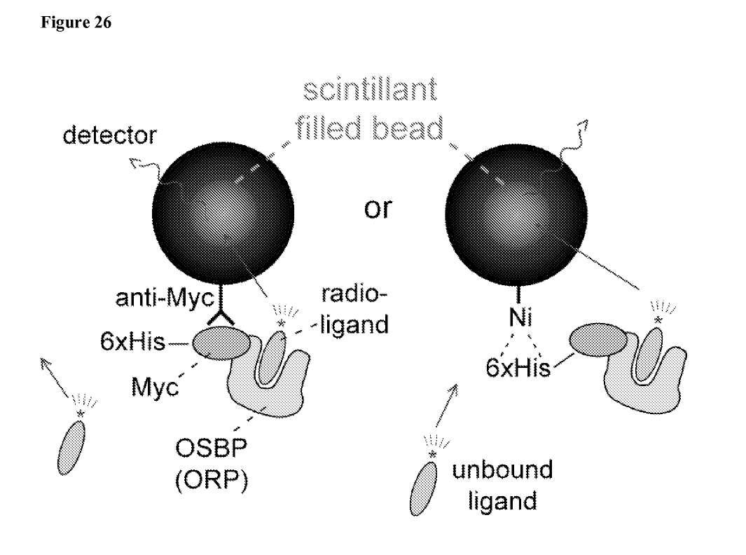

To measure the Kᵢs of CRAMs to OSBP and ORP4 reported above, a previously reported dextran-coated charcoal assay [51] was adapted into a 96-well format. However, this assay system does not work with purified protein, since crude lysate is required to maintain solubility of ³H-25-hydroxycholesterol and ³H-cholesterol ligands; and it is expensive and unwieldy to perform in the high-throughput format. To solve these limitations a binding SPA (scintillation proximity assay) for OSBP/ORPs (FIG. 26) will be developed.

First, cDNAs for the ORPs other than OSBP and ORP4 will be obtained from a commercial source (Invitrogen, Ultimate ORF™ collection), and these cDNAs will be cloned into the pcDNA3.1 vector, which places a C-terminal myc-his tag on the protein. The successful cloning of OSBP and ORP4 using this system has been performed. The various ORPs will be over-expressed in HEK-293T cells, as performed previously, and expression will be confirmed using western blot with a myc antibody.

As an initial screen for binding, ORP-myc-his S100 lysate (for ORPs 1-11) from HEK-293T cells will be exposed to polyvinyltoluene SPA beads coated with nickel or copper to bind the His-tag of ORPs. Or, alternatively, SPA beads conjugated to myc antibody will be used. This will bind the overexpressed ORP to the SPA bead (FIG. 26). The SPA beads contain the scintillant that only emits photons when radioligands are bound to the conjugated protein. Unbound ligands do not transfer energy and induce photon emission. By use of the SPA beads capable of affixing the overexpressed ORP through the myc-his tag, binding curves for ³H-25-OHC, ³H-25-cholesterol, ³H-1, and ³H-2 will then be produced. The use of a binding SPA is well established for studying the interaction of oxysterols with LXR proteins [52], and this body of work will serve as the model in constructing the OSBP/ORP binding SPA. Upon detection of saturable, specific binding in the crude ORP lysate, with the SPA, a large-scale expression of the select ORPs will be performed in HEK-293T cells. The ORP-myc-his protein will be purified first via nickel resin followed by FPLC size-exclusion chromatography to homogeneity. Alternatively, the OSBP/ORP expression could be performed via baculovirus/insect cell expression. The Ultimate ORF™ clones are provided in the Gateway™ System, which allows for facile cloning into many different expression vectors, including the BaculoDirect™ His-tagged insect cell vector.

### INCORPORATION BY REFERENCE

The following references correspond to the numbers above in brackets. To the extent that these references, or any other reference directly cited above, provide exemplary procedural or other details supplementary to those set forth therein, they are specifically incorportated by reference. In addition, all of the U.S. patents and U.S. published patent applications cited herein are hereby incorporated by reference.
1. Pettit G R, Inoue M, Kamano Y, Herald D L, Arm C, Dufresne C, et al. Antineoplastic agents. 147. Isolation and structure of the powerful cell growth inhibitor cephalostatin 1. Journal of the American Chemical Society. 1988 Jan. 1; 110(6):2006-7.
2. Kubo S, Mimaki Y, Terao M, Sashida Y, Nikaido T. Acylated cholestane glycosides from the bulbs of Ornithogalum saundersiae. Phytochemistry. 1992; 31(11):3969-73.
3. Mimaki Y, Kuroda M, Kameyama A, Sashida Y. Cholestane glycosides with potent cytostatic activities on various tumor cells from Ornithogalum saundersiae bulbs. Bioorganic & Medicinal Chemistry Letters. 1997 Jan. 1; 7(5):633-6.
4. Komiya T, Fusetani N, Matsunaga S, Kubo A, Kaye F J, Kelley M J, et al. Ritterazine B, a new cytotoxic natural compound, induces apoptosis in cancer cells. Cancer Chemother Pharmacology. 2003 Mar. 1; 51(3):202-8.
5. Beutler J A, Shoemaker R H, Johnson T, Boyd M R. Cytotoxic geranyl stilbenes from Macaranga schweinfurthii. J Nat Prod. 1998 Dec. 1; 61(12):1509-12.
6. Tasdemir D, Mangalindan G C, Concepcion G P, Verbitski S M, Rabindran S, Miranda M, et al. Bioactive isomalabaricane triterpenes from the marine sponge Rhabdastrella globostellata. J Nat Prod. 2002 Feb. 1; 65(2):210-4.
7. Shoemaker R H. The NCI60 human tumour cell line anticancer drug screen. Nat Rev Cancer. 2006 Oct. 1; 6(10):813-23.
8. Beutler J A, Jato J G, Gordon C, Wiemer D F, Neighbors J D, Salcius M, et al. THE SCHWEINFURTHINS: Issues in development of a plant-derived anticancer lead In: Bogers R J C, L. E.; Lange, D., editor. Medicinal and Aromatic Plants; 2006. p. 301-9.
9. Rudy A, Lopez-Anton N, Dirsch V M, Vollmar A M. The cephalostatin way of apoptosis. J Nat Prod. 2008 Mar. 1; 71(3):482-6.
10. Rudy A. Unusual apoptotic signaling pathways in cancer cells induced by cephalostatin. Munich: University of Munich 2007.
11. Lopez-Anton N, Rudy A, Barth N, Schmitz L, Pettit G, Schulze-Osthoff K, et al. The Marine Product Cephalostatin 1 Activates an Endoplasmic Reticulum Stress-specific and Apoptosome-independent Apoptotic Signaling Pathway. Journal of Biological Chemistry. 2006 Sep. 6; 281(44):33078-86.
12. Dirsch V M, Müller I M, Eichhorst S T, Pettit G R, Kamano Y, Inoue M, et al. Cephalostatin 1 Selectively Triggers the Release of Smac/DIABLO and Subsequent Apoptosis that is Characterized by an Increased Density of the Mitochondrial Matrix. Cancer Research. 2003 Dec. 15; 63(24):8869-76.
13. Taghiyev A F, Guseva N V, Glover R A, Rokhlin O W, Cohen M B. TSA-induced cell death in prostate cancer cell lines is caspase-2 dependent and involves the PIDDosome. Cancer Biol Ther. 2006 Sep. 1; 5(9):1199-205.
14. Zhou Y. OSW-1: a Natural Compound With Potent Anticancer Activity and a Novel Mechanism of Action. J Natl Cancer Inst. 2005 Dec. 7; 97(23):1781-5.
15. Topczewski J J, Neighbors J D, Wiemer D F. Total Synthesis of (+)-Schweinfurthins B and E. The Journal of Organic Chemistry. 2009; 74(18):6965-72.
16. Mente N R, Wiemer A J, Neighbors J D, Beutler J A, Hohl R J, Wiemer D F. Total synthesis of (R,R,R)- and (S,S,S)-schweinfurthin F: differences of bioactivity in the enantiomeric series. Bioorganic & Medicinal Chemistry Letters. 2007 Feb. 15; 17(4):911-5.
17. Abukhdeir A M, Park B H. P21 and p27: roles in carcinogenesis and drug resistance. Expert reviews in molecular medicine. 2008 Jan. 1; 10:e19.
18. Somlo G, Chu P, Frankel P, Ye W, Groshen S, Doroshow J H, et al. Molecular profiling including epidermal growth factor receptor and p21 expression in high-risk breast cancer patients as indicators of outcome. Ann Oncol. 2008 Nov. 1; 19(11):1853-9.
19. Gartel A L, Tyner A L. The role of the cyclin-dependent kinase inhibitor p21 in apoptosis. Mol Cancer Ther. 2002 Jun. 1; 1(8):639-49.
20. Olkkonen V M, Johansson M, Suchanek M, Yan D, Hynynen R, Ehnholm C, et al. The OSBP-related proteins (ORPs): global sterol sensors for co-ordination of cellular lipid metabolism, membrane trafficking and signalling processes? Biochem Soc Trans. 2006 Jun. 1; 34(Pt 3):389-91.
21. Yan D, Olkkonen V M. Characteristics of oxysterol binding proteins. Int Rev Cytol. 2008 Jan. 1; 265:253-85.
22. Ridgway N D. 25-Hydroxycholesterol stimulates sphingomyelin synthesis in Chinese hamster ovary cells. J Lipid Res. 1995 Jun. 1, 1995; 36(6):1345-58.
23. Zeidan Y, Hannun Y A. The Acid/Sphingomyelin/Ceramide Pathway: Biomedical Significance and Mechanism of Regulation. Current Molecular Medicine. 2009; Epub aheda of print.
24. Perry R J, Ridgway N D. Oxysterol-binding protein and vesicle-associated membrane protein-associated protein are required for sterol-dependent activation of the ceramide transport protein. Mol Biol Cell. 2006 Jun. 1; 17(6):2604-16.
25. Im Y, Raychaudhuri S, Prinz W, Hurley J. Structural mechanism for sterol sensing and transport by OSBP-related proteins. Nature. 2005 Sep. 1; 437(7055):154-8.
26. Wang P-y, Liu P, Weng J, Sontag E, Anderson R G W. A cholesterol-regulated PP2A/HePTP complex with dual specificity ERK1/2 phosphatase activity. EMBO J. 2003; 22(11):2658-67.
27. Wang P Y, Weng J, Anderson R G. OSBP is a cholesterol-regulated scaffolding protein in control of ERK 1/2 activation. Science. 2005 Mar. 4; 307(5714):1472-6.
28. Yoon S, Seger R. The extracellular signal-regulated kinase: multiple substrates regulate diverse cellular functions. Growth Factors. 2006 Mar. 1; 24(1):21-44.
29. Devarajan E, Huang S. STAT3 as a Central Regulator of Tumor Metastases. Current Molecular Medicine. 2009; 9(5):626-33.
30. Svensson S, Ostberg T, Jacobsson M, Norstrom C, Stefansson K, Hallen D, et al. Crystal structure of the heterodimeric complex of LXR[alpha] and RXR[beta] ligand-binding domains in a fully agonistic conformation. EMBO J. 2003; 22(18):4625-33.
31. Radhakrishnan A, Ikeda Y, Kwon H J, Brown M S, Goldstein J L. Sterol-regulated transport of SREBPs from endoplasmic reticulum to Golgi: oxysterols block transport by binding to Insig. Proc Natl Acad Sci USA. 2007 Apr. 17; 104(16):6511-8.
32. Kwon H J, Abi-Mosleh L, Wang M L, Deisenhofer J, Goldstein J L, Brown M S, et al. Structure of N-Terminal Domain of NPC1 Reveals Distinct Subdomains for Binding and Transfer of Cholesterol. Cell. 2009; 137(7):1213-24.
33. Yan D, Jauhiainen M, Hildebrand R, Willems Van Dijk K, Van Berkel T, Ehnholm C, et al. Expression of Human OSBP-Related Protein 1 L in Macrophages Enhances Atherosclerotic Lesion Development in LDL Receptor-Deficient Mice. Arteriosclerosis, Thrombosis, and Vascular Biology. 2007 Jul. 1; 27(7):1618-24.
34. Yan D, Mayranpaa M I, Wong J, Perttila J, Lehto M, Jauhiainen M, et al. OSBP-related Protein 8 (ORP8) Suppresses ABCA1 Expression and Cholesterol Efflux from Macrophages. J Biol Chem. 2008 Jan. 4, 2008; 283(1):332-40.
35. Bowden K, Ridgway N, OSBP Negatively Regulates ABCA1 Protein Stability. Journal of Biological Chemistry. 2008 Apr. 22; 283(26):18210-7.
36. Fournier M V, Guimaraes F C, Paschoal M E M, Ronco L V, Carvalho MdGC, Pardee A B. Identification of a Gene Encoding a Human Oxysterol-binding Protein-Homologue: A Potential General Molecular Marker for Blood Dissemination of Solid Tumors. Cancer Res. 1999 Aug. 1, 1999; 59(15):3748-53.
37. Koga Y, Ishikawa S, Nakamura T, Masuda T, Nagai Y, Takamori H, et al. Oxysterol binding protein-related protein-5 is related to invasion and poor prognosis in pancreatic cancer. Cancer Science. 2008 Dec. 1; 99(12):2387-94.
38. Warner-Lambert X G, inventor ORP9, A Novel Therapeutic Target For Increasing HDL Levels. 2004.
39. Michael L F, Schkeryantz J M, Burris T P. The Pharmacology of LXR. Mini-Reviews in Medicinal Chemistry. 2005; 5(8):729-40.
40. Beh C T, Cool L, Phillips J, Rine J. Overlapping Functions of the Yeast Oxysterol-Binding Protein Homologues. Genetics. 2001 Mar. 1, 2001; 157(3):1117-40.
41. Ferrandiz N, Martin-Perez J, Blanco R, Donertas D, Weber A, Eilers M, et al. HCT116 Cells Deficient in p21(Waf1) Are Hypersensitive to Tyrosine Kinase Inhibitors and Adriamycin Through a Mechanism Unrelated to p21 and Dependent on p53. DNA Repair. 2009; 8(3):390-9.
42. Abbas T, Dutta A. p21 in cancer: intricate networks and multiple activities. Nat Rev Cancer. 2009 Jun. 14; 9(6):400-14.
43. Dawson P A, Van der Westhuyzen D R, Goldstein J L, Brown M S. Purification of oxysterol binding protein from hamster liver cytosol. J Biol Chem. 1989 May 25; 264(15):9046-52.
44. Perry R J, Ridgway N D. Molecular mechanisms and regulation of ceramide transport. Biochim Biophys Acta. 2005 Jun. 1; 1734(3):220-34.
45. Carpinteiro A, Dumitru C, Schenck M, Gulbins E. Ceramide-induced cell death in malignant cells. Cancer Letters. 2008 Jun. 8; 264(1):1-10.
46. Ridgway N D, Dawson P A, Ho Y K, Brown M S, Goldstein J L. Translocation of oxysterol binding protein to Golgi apparatus triggered by ligand binding. The Journal of Cell Biology. 1992 Jan. 1; 116(2):307-19.
47. Fortner K C, Kato D, Tanaka Y, Shair M D. Enantioselective Synthesis of Cephalostatin 1. Journal of the American Chemical Society. 2009; Dec. 7. [Epub ahead of print]
48. Xue J, Liu P, Pan Y, Guo Z. A total synthesis of OSW-1. J Org Chem. 2008 Jan. 4; 73(1):157-61.
50. Orita A, Sakamoto K, Hamada Y, Mitsutome A, Otera J. Mild and Practical Acylation of Alcohols with Esters or Acetic Anhydride Under Distannoxane Catalysis Tetrahedron. 1999; 55(10):2899-910.
51. Kandutsch A A, Taylor F R, Shown E P. Different forms of the oxysterol-binding protein. Binding kinetics and stability. J Biol Chem. 1984 Oct. 25; 259(20):12388-97.
52. Janowski B A, Grogan M J, Jones S A, Wisely G B, Kliewer S A, Corey E J, et al. Structural requirements of ligands for the oxysterol liver X receptors LXRÎ± and LXRÎ². Proceedings of the National Academy of Sciences of the United States of America. 1999 Jan. 5, 1999; 96(1):266-71.

### EQUIVALENTS

The invention has been described broadly and generically herein. Those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention. Further, each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

1. A method of treating an oxysterol binding protein-related disease or condition, comprising administering to a subject in need thereof an effective amount of a compound of formula I represented by 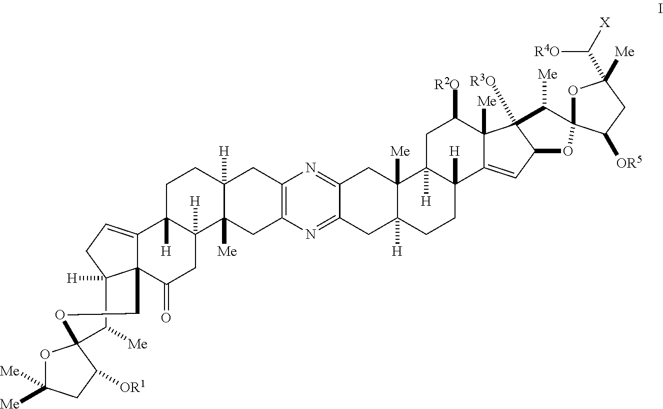
or a pharmaceutically acceptable salt, biologically active metabolite, solvate, hydrate, prodrug, enantiomer or stereoisomer thereof, wherein, independently for each occurrence,
R¹ is hydrogen, silyl, aralkyl, alkylcarboxy, arylcarboxy, heteroarylcarboxy, aralkylcarboxy or heteroaralkylcarboxy;
R² is hydrogen, silyl, aralkyl, alkylcarboxy, arylcarboxy, heteroarylcarboxy, aralkylcarboxy or heteroaralkylcarboxy;
R³ is hydrogen, silyl, aralkyl, alkylcarboxy, arylcarboxy, heteroarylcarboxy, aralkylcarboxy or heteroaralkylcarboxy;
R⁴ is hydrogen, silyl, aralkyl, alkylcarboxy, arylcarboxy, heteroarylcarboxy, aralkylcarboxy or heteroaralkylcarboxy;
R⁵ is hydrogen, silyl, aralkyl, alkylcarboxy, arylcarboxy, heteroarylcarboxy, aralkylcarboxy or heteroaralkylcarboxy; and
X is hydrogen, deuterium, tritium, alkyl, aralkyl or heteroaralkyl.

2. A method of treating an oxysterol binding protein-related disease or condition, comprising administering to a subject in need thereof an effective amount of a compound of formula II represented by 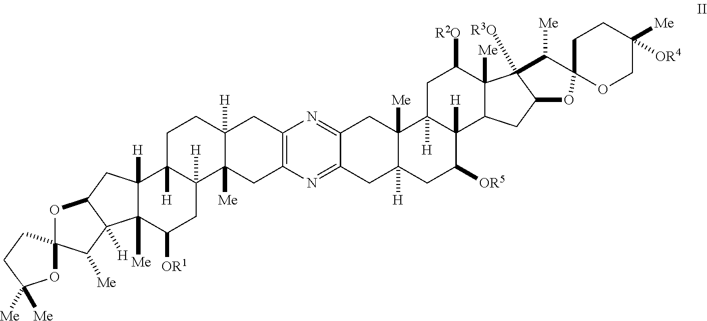
or a pharmaceutically acceptable salt, biologically active metabolite, solvate, hydrate, prodrug, enantiomer or stereoisomer thereof, wherein, independently for each occurrence,
R¹ is hydrogen, silyl, aralkyl, alkylcarboxy, arylcarboxy, heteroarylcarboxy, aralkylcarboxy or heteroaralkylcarboxy;
R² is hydrogen, silyl, aralkyl, alkylcarboxy, arylcarboxy, heteroarylcarboxy, aralkylcarboxy or heteroaralkylcarboxy;
R³ is hydrogen, silyl, aralkyl, alkylcarboxy, arylcarboxy, heteroarylcarboxy, aralkylcarboxy or heteroaralkylcarboxy;
R⁴ is hydrogen, silyl, aralkyl, alkylcarboxy, arylcarboxy, heteroarylcarboxy, aralkylcarboxy or heteroaralkylcarboxy; and
R⁵ is hydrogen, silyl, aralkyl, alkylcarboxy, arylcarboxy, heteroarylcarboxy, aralkylcarboxy or heteroaralkylcarboxy.

3. A method of treating an oxysterol binding protein-related disease or condition, comprising administering to a subject in need thereof an effective amount of a compound of formula III represented by 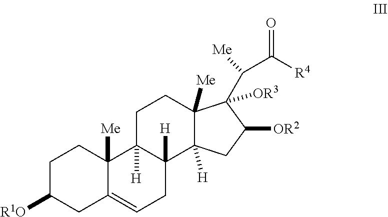
or a pharmaceutically acceptable salt, biologically active metabolite, solvate, hydrate, prodrug, enantiomer or stereoisomer thereof, wherein, independently for each occurrence,
R¹ is hydrogen, silyl, aralkyl, alkylcarboxy, arylcarboxy, heteroarylcarboxy, aralkylcarboxy or heteroaralkylcarboxy;
R² is hydrogen or a carbohydrate;
R³ is hydrogen, silyl, aralkyl, alkylcarboxy, arylcarboxy, heteroarylcarboxy, aralkylcarboxy or heteroaralkylcarboxy; and
R⁴ is alkyl, aralkyl or heteroaralkyl.

4. The method of claim 3, wherein R² is 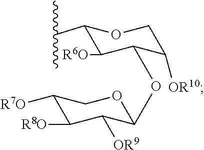
and
R⁶, R⁷, R⁸, R⁹ and R¹⁰ are independently selected from the group consisting of hydrogen, silyl, aralkyl, alkylcarboxy, arylcarboxy, heteroarylcarboxy, aralkylcarboxy and heteroaralkylcarboxy.

5. A method of treating an oxysterol binding protein-related disease or condition, comprising administering to a subject in need thereof an effective amount of a compound of formula IV represented by 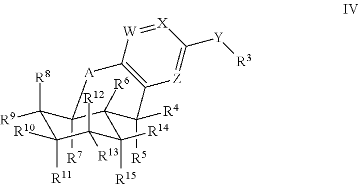
or a pharmaceutically acceptable salt, biologically active metabolite, solvate, hydrate, prodrug, enantiomer or stereoisomer thereof, wherein, independently for each occurrence,
A is —O—, —S(═O)ₙ—, —C(R^{C})₂—, or —N(R^{N})—;
n is 0, 1 or 2;
W is —C(R¹)— or —N—;
X is —C(R²)— or —N—;
Y is -alkylene-R;
Z is —C(R³)— or —N—;
R is an optionally substituted prenylaryl or prenylheteroaryl;
R¹, R¹¹ and R¹³ are independently selected from the group consisting of alkenyl, alkoxy, alkyl, alkylcarbonyloxy, alkylthio, alkylcarbonylthio, alkynyl, amido, amidoalkyl, amino, aminoalkyl, carboxy, cyano, formyl, halo, haloalkoxy, haloalkyl, haloalkylthio, hydroxyl, hydroxyalkyl, mercapto, mercaptoalkyl, nitro, silyl, silyloxy and silyloxyalkyl;
R², R³, R⁴, R⁵, R⁸, R⁹, R¹⁰, R¹² and R^{C} are independently selected from the group consisting of alkenyl, alkoxy, alkyl, alkylcarbonyloxy, alkylthio, alkylcarbonylthio, alkynyl, amido, amidoalkyl, amino, aminoalkyl, carboxy, cyano, formyl, halo, haloalkoxy, haloalkyl, haloalkylthio, hydrogen, hydroxyl, hydroxyalkyl, mercapto, mercaptoalkyl, nitro, silyl, silyloxy and silyloxyalkyl;
R⁶, R⁷, R¹⁴ and R¹⁵ are independently selected from the group consisting of hydrogen, alkyl and haloalkyl; and
R^{N} is hydrogen, alkyl, alkylcarbonyl, aralkylcarbonyl or haloalkyl.

6. A method of treating an oxysterol binding protein-related disease or condition, comprising administering to a subject in need thereof an effective amount of a compound of formula V represented by 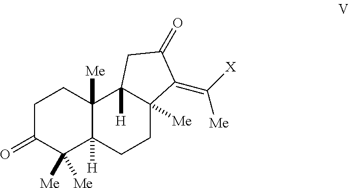
or a pharmaceutically acceptable salt, biologically active metabolite, solvate, hydrate, prodrug, enantiomer or stereoisomer thereof, wherein, independently for each occurrence,
X is 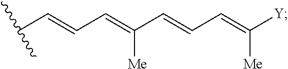
Y is —CH₂OR¹, —C(═O)R², —C(═O)OR³ or —C(═O)NHR³;
R¹ is hydrogen, silyl, aralkyl, alkylcarboxy, arylcarboxy, heteroarylcarboxy, aralkylcarboxy or heteroaralkylcarboxy;
R² is hydrogen, alkyl, aryl, heteroaryl, aralkyl or heteroaralkyl; and
R³ is hydrogen, alkyl, aryl, heteroaryl, aralkyl, heteroaralkyl, alkylcarboxy, arylcarboxy, heteroarylcarboxy, aralkylcarboxy or heteroaralkylcarboxy.

7-11. (canceled)

12. The method of claim 5, wherein R is substituted with between one and ten substituents independently selected from the group consisting of alkenyl, alkoxy, alkyl, alkylcarbonyloxy, alkylthio, alkylcarbonylthio, alkynyl, amido, amidoalkyl, amino, aminoalkyl, carboxy, cyano, formyl, halo, haloalkoxy, haloalkyl, haloalkylthio, hydroxyl, hydroxyalkyl, mercapto, mercaptoalkyl, nitro, silyl, silyloxy and silyloxyalkyl.

13. The method of any one of claims 1-6 and 12, wherein the oxysterol binding protein is oxysterol binding protein 1 (OSBP1).

14. The method of any one of claims 1-6 and 12, wherein the oxysterol binding protein-related disease or condition is atherosclerosis.

15. The method of any one of claims 1-6 and 12, wherein the oxysterol binding protein-related disease or condition is Alzheimer's disease.

16. The method of any one of claims 1-6 and 12, wherein the oxysterol binding protein-related disease or condition is cancer.

17. The method of claim 16, wherein the cancer is a p21-deficient cancer.
